SEQUENCE LISTING (41 sequences)

SEQ ID NO: 1 - protein, 19 aa, Homo sapiens
YSMKETTMKIIPFNRLTIG

SEQ ID NO: 2 - DNA, 378 bp, synthetic construct
  source 1..378: mol_type = other DNA
gaagtgcagctggtggagtctgggggaggcgcggtccaacctgggaggtccctgagactctcctgtgcagcctctggagtcagtttcagtaacattggcatgcactgggtccgccaggctccaggcaaggggctggagtgggtggcacttatatcatctgatggacgtcatacacactatgcagactccgtgaagggccgattcaccatctccagagacaattccgagaacacgctctatctacagatcaacggcctgagagctgacgacacggctgtttattactgtgcgaaaggccttgacactcgtgcccgttacatggggaaattttatacttttgactactggggccagggaaccctggtcaccgtctcctca

SEQ ID NO: 3 - protein, 126 aa, Homo sapiens
EVQLVESGGGAVQPGRSLRLSCAASGVSFSNIGMHWVRQAPGKGLEWVALISSDGRHTHYADSVKGRFTISRDNSENTLYLQINGLRADDTAVYYCAKGLDTRARYMGKFYTFDYWGQGTLVTVSS

SEQ ID NO: 4 - DNA, 321 bp, synthetic construct
  source 1..321: mol_type = other DNA
gacatccagttgacccagtctccatcctccctgtctgcatctgtaggagacagagtcaccatcacttgtcgggcaagtcagaacattggcgtcaatttaaattggtatcaacagagaccagggaaaggcccgaaactcctagtctcttctacatttagtttgcaaagtggggtcccatcaaggtttagtgccagtggagctgggacaaatttcactctcaccatcagcagtctgcaacctgaggactatgtcacttactactgtcaacagagttacagtagtccattcacattcggccctgggaccaaagtggatatcaaa

SEQ ID NO: 5 - protein, 107 aa, Homo sapiens
DIQLTQSPSSLSASVGDRVTITCRASQNIGVNLNWYQQRPGKGPKLLVSSTFSLQSGVPSRFSASGAGTNFTLTISSLQPEDYVTYYCQQSYSSPFTFGPGTKVDIK

SEQ ID NO: 6 - protein, 15 aa, Homo sapiens
FKSQLQKVPPEWKAL

SEQ ID NO: 7 - protein, 15 aa, Homo sapiens
FPGFFSPMLGEFVSE

SEQ ID NO: 8 - protein, 15 aa, Homo sapiens
FSPMLGEFVSETESR

SEQ ID NO: 9 - protein, 15 aa, Homo sapiens
RGHAKSRPVRGIHTS

SEQ ID NO: 10 - protein, 15 aa, Homo sapiens
GGCLHADPDLGVLCP

SEQ ID NO: 11 - protein, 15 aa, Homo sapiens
DLGVLCPTGCQLQEA

SEQ ID NO: 12 - protein, 15 aa, Homo sapiens
VSQTSSSSFQYMYLL

SEQ ID NO: 13 - protein, 15 aa, Homo sapiens
VNEYSSELEKHQLYI

SEQ ID NO: 14 - protein, 15 aa, Homo sapiens
EYCRTPCTVSCNIPV

SEQ ID NO: 15 - protein, 15 aa, Homo sapiens
PYRVYCDMNTENGGW

SEQ ID NO: 16 - protein, 15 aa, Homo sapiens
SVDFGRKWDPYKQGF

SEQ ID NO: 17 - protein, 15 aa, Homo sapiens
KNYCGLPGEYWLGND

SEQ ID NO: 18 - protein, 15 aa, Homo sapiens
EDWKGDKVKAHYGGF

SEQ ID NO: 19 - protein, 15 aa, Homo sapiens
GDKVKAHYGGFTVQN

SEQ ID NO: 20 - protein, 15 aa, Homo sapiens
RKQCSKEDGGGWWYN

SEQ ID NO: 21 - protein, 15 aa, Homo sapiens
SKEDGGGWWYNRCHA

SEQ ID NO: 22 - protein, 15 aa, Homo sapiens
CHAANPNGRYYWGGQ

SEQ ID NO: 23 - protein, 15 aa, Homo sapiens
RYYWGGQYTWDMAKH

SEQ ID NO: 24 - protein, 15 aa, Homo sapiens
AKHGTDDGVVWMNWK

SEQ ID NO: 25 - protein, 15 aa, Homo sapiens
NCCILDERFGSYCPT

SEQ ID NO: 26 - protein, 15 aa, Homo sapiens
FGSYCPTTCGIADFL

SEQ ID NO: 27 - protein, 15 aa, Homo sapiens
NLKEKVAQLEAQCQE

SEQ ID NO: 28 - protein, 15 aa, Homo sapiens
DFKKNWIQYKEGFGH

SEQ ID NO: 29 - protein, 15 aa, Homo sapiens
NWIQYKEGFGHLSPT

SEQ ID NO: 30 - protein, 15 aa, Homo sapiens
FGHLSPTGTTEFWLG

SEQ ID NO: 31 - protein, 15 aa, Homo sapiens
GDAFDGFDFGDDPSD

SEQ ID NO: 32 - protein, 15 aa, Homo sapiens
FFTSHNGMQFSTWDN

SEQ ID NO: 33 - protein, 15 aa, Homo sapiens
WMNKCHAGHLNGVYY

SEQ ID NO: 34 - protein, 15 aa, Homo sapiens
GSGWWMNKCHAGHLN

SEQ ID NO: 35 - protein, 15 aa, Homo sapiens
WMNKCHAGHLNGVYY

SEQ ID NO: 36 - protein, 15 aa, Homo sapiens
DFKKNWIQYKEGFGH

SEQ ID NO: 37 - protein, 15 aa, Homo sapiens
FPGFFSPMLGEFVSE

SEQ ID NO: 38 - DNA, 1374 bp, Mus musculus
  source 1..1374: mol_type = other DNA
gaagtgcagctggtggaatctggtggcggagctgttcagcctggcagatccctgagactgtcttgtgctgcttccggcgtgtccttctccaacatcggcatgcactgggtccgacaggcccctggaaaaggattggaatgggtcgccctgatctcctctgacggcagacacacccactacgccgactctgtgaagggcagattcaccatcagccgggacaactccgagaacaccctgtacctgcagatcaacggcctgagagccgatgacaccgccgtgtactactgtgccaaaggcctggatacccgcgctcggtacatgggcaagttctacaccttcgactactggggccagggcaccctggttacagtttcttctgctaagaccaccgcacctagtgtgtaccctctggctcctgtgtgtggcgataccacaggctcctctgtgaccctgggatgtctggtcaagggctacttccccgagcctgtgacactgacctggaactccggatctctgtcctccggcgtgcacacctttccagctgtgctgcagtccgacctgtacaccctgtccagctctgtgactgtgacctcctccacctggcctagccagtccatcacctgtaacgtggcccatcctgcctccagcaccaaggtggacaagaagatcgagcctcggggccctaccatcaagccttgtcctccatgcaagtgccccgctcctaatctgctcggaggcccttccgtgttcatcttcccacctaagatcaaggacgtgctgatgatctccctgtctcctatcgtgacctgcgtggtggtggacgtgtccgaggatgatcctgacgtgcagattagttggttcgtgaacaacgtcgaggtgcacaccgctcagacccagacacacagagaggactacaactccacactgagagtggtgtctgccctgcctatccagcatcaggattggatgtccggcaaagaattcaagtgcaaagtgaacaacaaggacctgcctgctccaatcgagcggaccatctctaagcctaagggctctgtcagggcccctcaggtgtacgttttgccacctcctgaggaagagatgaccaagaaacaagtgaccctgacatgcatggtcaccgacttcatgcccgaggacatctacgtggaatggaccaacaacggcaagaccgagctgaattacaagaacacagagcccgtgctggactccgacggctcctacttcatgtactccaagctgcgcgtcgagaagaagaactgggtcgagagaaactcctactcctgctccgtggtgcacgagggcctgcacaatcaccacaccaccaagtccttctctcggacccctggaaagtgatga

SEQ ID NO: 39 - protein, 456 aa, Mus musculus
EVQLVESGGGAVQPGRSLRLSCAASGVSFSNIGMHWVRQAPGKGLEWVALISSDGRHTHYADSVKGRFTISRDNSENTLYLQINGLRADDTAVYYCAKGLDTRARYMGKFYTFDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 40 - DNA, 648 bp, Mus musculus
  source 1..648: mol_type = other DNA
gatatccagctgacccagtctccttccagcctgtctgcctctgtgggcgacagagtgaccatcacctgtcgggcctctcagaacatcggcgtgaacctgaactggtatcagcagaggcctggcaagggccctaaactgctggtgtcctctaccttcagcctgcagtctggcgtgccctccagattttctgcttctggcgctggcaccaactttaccctgacaatcagctccctgcagcctgaggactacgtgacctactactgccagcagtcctactcctctccattcaccttcggacccggcaccaaggtggacattaagagagctgacgccgctcctaccgtgtctatcttcccaccctctagcgagcagctgacctcaggcggagcttctgtcgtgtgcttcctgaacaacttctaccccaaggacatcaacgtgaagtggaagatcgacggctccgagagacagaacggcgtgctgaactcttggaccgaccaggactccaaggacagcacctactccatgtcctccacactgaccctgaccaaggacgagtacgagcggcacaactcctatacctgcgaggctacccacaagacctccacctctccaatcgtgaagtccttcaaccggaacgagtgctgatga

SEQ ID NO: 41 - protein, 214 aa, Mus musculus
DIQLTQSPSSLSASVGDRVTITCRASQNIGVNLNWYQQRPGKGPKLLVSSTFSLQSGVPSRFSASGAGTNFTLTISSLQPEDYVTYYCQQSYSSPFTFGPGTKVDIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

(19) United States
(12) Patent Application Publication
(10) Pub. No.: US 2026/0146103 A1
(43) Pub. Date: May 28, 2026

(54) HUMAN ANTI-FIBRIN ANTIBODIES AND USES THEREOF

(71) Applicants: The J. David Gladstone Institutes, a testamentary trust established under the Will of J. David, San Francisco, CA (US); The Regents of the University of California, Oakland, CA (US)

(72) Inventors: Katerina Akassoglou, San Francisco, CA (US); Hans-Christian von Buedingen, Basel (CH); Jae Kyu Ryu, San Francisco, CA (US)

(21) Appl. No.: 19/105,455

(22) PCT Filed: Aug. 22, 2023
(86) PCT No.: PCT/US2023/072642

§ 371 Date: Feb. 21, 2025

Related U.S. Application Data
(60) Provisional application No. 63/399,752, filed on Aug. 22, 2022.

(51) Int. Cl. C07K 16/36; A61K 39/00; A61P 37/06

(52) U.S. Cl. CPC C07K 16/36; A61P 37/06; A61K 2039/505; C07K 2317/33; C07K 2317/92

(57) ABSTRACT

Provided herein are anti-fibrin antibodies and methods of their use.

### PRIORITY

This application claims the benefit of priority of U.S. Provisional Application No. 63/399,752, filed Aug. 22, 2022, the contents of which are incorporated by reference herein in its entirety for any purpose.

### GOVERNMENT SUPPORT

This invention was made with government support under grant no. NS052189 awarded by The National Institutes of Health. The government has certain rights in the invention.

### SEQUENCE LISTING

This application contains a Sequence Listing which has been submitted electronically in ST26 format and is hereby incorporated by reference in its entirety. Said ST26 file, created on Aug. 22, 2023, is named “3730195WO1.xml” and is 40,061 bytes in size.

### BACKGROUND

Fibrinogen is a glycoprotein complex, produced in the liver, that circulates in the blood of all vertebrates. During tissue and vascular injury, it is converted enzymatically by thrombin to fibrin and then to a fibrin-based blood clot. Fibrin clots function primarily to occlude blood vessels to stop bleeding. Fibrin also binds and reduces the activity of thrombin.

### SUMMARY

Provided herein is a human antibody comprising a heavy chain variable domain (VH) comprising the amino acid sequence of SEQ ID NO:3 or at least 95% identity thereto and a light chain variable domain (VL) comprising the amino acid sequence of SEQ ID NO:5 or at least 95% identity thereto. In one embodiment, the antibody can bind fibrin and/or fibrinogen. In one embodiment, the antibody binds the fibrin P2 (SEQ ID NO: 1) epitope. In one embodiment, the antibody binds one or more of SEQ ID NOs: 1, 6, 13, 17, 22, 25, 31, 32, 34, 35, 36 and/or 37.

In one embodiment, the antibody inhibits proinflammatory properties of fibrin in macrophages. In one embodiment, the antibody does not affect blood coagulation. One embodiment provides a composition comprising an antibody described herein and a carrier.

One embodiment provides a method to treat or prevent a neurodegenerative disease or disorder comprising administering to a subject in need thereof an effective amount of an antibody or composition described herein. In one embodiment, the neurodegenerative disease or disorder is selected from the group consisting of multiple sclerosis (MS), progressive multifocal leukoencephalopathy (PML), encephalomyelitis (EPL), central pontine myelolysis (CPM), adrenoleukodystrophy, Alexander's disease, Pelizaeus Merzbacher disease (PMZ), Wallerian Degeneration, optic neuritis, transverse myelitis, amyotrophic lateral sclerosis (ALS), Huntington's disease, Alzheimer's disease, Parkinson's disease, spinal cord injury, traumatic brain injury, post radiation injury, neurologic complications of chemotherapy, stroke, acute ischemic optic neuropathy, vitamin E deficiency, isolated vitamin E deficiency syndrome, AR, Bassen-Kornzweig syndrome, Marchiafava-Bignami syndrome, metachromatic leukodystrophy, trigeminal neuralgia, acute disseminated encephalitis, Guillian-Barre syndrome, Marie-Charcot-Tooth disease and Bell's palsy.

One embodiment provides a method to treat a condition/disease in which fibrin plays a role comprising administering to a subject in need thereof an effective amount of an antibody or composition described herein. In one embodiment, the condition/disease in which fibrin plays a role comprises inflammation, periodontal disease, rheumatoid arthritis (RA), cancer (e.g., B cell lymphoma), colitis or muscular dystrophy.

Another embodiment provides a method comprising administering to a subject in need thereof an effective amount of an antibody or the composition provided herein, wherein the subject is infected with SARS-CoV-2 or SARS-CoV-1.

One embodiment provides a method to inhibit microglial activity/activation comprising administering to a subject in need thereof an effective amount of an antibody or composition described herein.

One embodiment provides a method to diagnose a fibrin-related disease or disorder comprising: (a) contacting the antibody described herein with a sample from a subject under conditions that allow polypeptide/antibody complexes to form; and (b) detecting polypeptide/antibody complexes of a), wherein the detection of polypeptide/antibody complexes is an indication that fibrin is present in the sample.

One aspect provides a method to promote remyelination comprising administering to a subject in need thereof an effective amount of an antibody or composition provided herein.

One aspect provides a method to protect a subject from myelin damage or inhibit myelin damage in a subject comprising administering to said subject the antibody or fragment thereof or the composition as disclosed herein, wherein the myelin damage is due to an inflammatory demyelinating disease.

Another embodiment provides a kit comprising the antibody or composition described herein.

Additional objects and advantages will be set forth in part in the description which follows, and in part will be understood from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one or more embodiment(s) and together with the description, serve to explain the principles described herein.

### BRIEF DESCRIPTION OF THE DRAWINGS

FIGS. 1A-1D demonstrates antibody binding to fibrinogen and fibrin (antibodies A-R).

FIG. 2 demonstrates that antibody G (45-C1C3) does not inhibit fibrin polymerization.

FIG. 3 demonstrates that antibody G (45-C1C3) reduces fibrin induced chemokine expression.

FIG. 4 provides double immunofluorescence with a sheep anti-fibrin(ogen) antibody (Enzyme Research) (red) and antibody G (green) shows antibody G recognition of fibrin(ogen) in the spinal cord. All images captured at 10×. Merge is also shown at 20×.

FIG. 5 provides a representative flow cytometry of CD19+CD138+ plasmablasts/plasma cells from cerebrospinal fluid.

FIGS. 6A-B provide (A) Antibody-G binding in a dose response curve on 25 μg/mL IgG-depleted fibrinogen with a calculated Kd of 0.66 μM. (B) Antibody-G binding on 25 μg/mL IgG-depleted fibrin with a Kd of 0.16 μM.

FIG. 7 depicts Antibody G (Ab G) epitope profiling performed with PepStar™ Multiwell peptide microarray containing 390 individual peptides that span fibrinogen alpha (FIBA), beta (FIBB) and gamma chains (FIBG). Scale is in RLUs converted to log base of 10.

FIG. 8 provides a list of Antibody G epitopes derived from the fibrinogen peptide array. 15mer peptide sequences that resulted in greater than 2500 relative light units (RLU) are shown with corresponding signal intensity.

FIG. 9 provides list of Antibody G epitopes identified by fibrinogen peptide array.

FIG. 10 provides the relative binding of 25 and 50 μg/mL Antibody G to 10 candidate peptides (30 μM each). Graph is representative of two independent experiments.

FIG. 11 demonstrates (Left) Antibody G binding on 30 μM FIBG331-345 peptide with a calculated Kd of 0.36 μM. (Right) Antibody G binding on 30 μM P2 peptide with a calculated Kd of 11.8 μM.

FIG. 12 depicts immunofluorescent detection of Antibody G binding to target in vivo. Microscopy of spinal cord sections from MOG35-55 EAE mouse after intraperitoneal administration of biotinylated Antibody G detected by Streptavidin-FITC (green), co-labeled with fibrin (red) and DAPI.

FIG. 13 depicts immunofluorescent detection of Antibody-G binding in EAE, spinal cord lesions. Microscopy of spinal cord sections from MOG35-ss EAE stained with Fibrin (red), Antibody G (green) and DAPI.

FIG. 14 demonstrates changes in ROS production induced by the treatment of fibrin-stimulated BMDMs with Antibody G. Quantification of ROS production (assessed via DHE) in mouse bone-marrow derived macrophages left unstimulated or stimulated for 24 h with fibrin in the presence of Antibody G (Ab-G) or hIgG1, presented in arbitrary units (AU). Data from three independent experiments (mean+s.e.m.). **P<0.01, ***P<0.001, ****P<0.0001 (one-way ANOVA with Tukey's multiple comparisons test).

FIGS. 15A-15B provide data that MS-derived autoantibody blocks fibrinogen-induced microglial activation and macrophage infiltration. Microscopy images of brain sections showing corpus callosum 3 d after fibrinogen injection in mice treated with stereotactic i.c.v. injection of 20 ug Antibody G (Ab-G) or hIgG1, tissues were stained for Iba-1 (A: a marker for microglial activation) or Mac-2 (B: a marker for macrophages). Quantification of Iba-1 and Mac-2 immunoreactivity in the corpus callosum 3 days after injection of fibrinogen in the corpus callosum of mice treated with stereotactic i.c.v. injection of Antibody G or hIgG1 (right bar graphs). Data are mean±s.e.m; ACSF, n=6 mice, Fibrinogen, n=6 mice, Fibrinogen-+Ab-G, n=6 mice, Fibrinogen+hIgG1, n=6 mice. *P<0.05, **P<0.01, ***P<0.001, by one-way ANOVA with Tukey's multiple comparisons test.

FIG. 16 provides solid phase ELISA of murine antibody G binding to fibrin.

FIG. 17 demonstrates that multiple sclerosis patient-derived autoantibody murine Antibody-G suppresses EAE. EAE was induced on day 0 using MOG35-55, followed by therapeutic administration of MS autoantibody murine Antibody-G (mu-AbG, 40 mg/kg) or IgG2a (40 mg/kg) every 3 d from day 3. Data are from n=10 mice (mu-AbG) or n=10 mice (IgG2a).

FIG. 18 demonstrates that therapeutic treatment with mu-AbG antibody reduces from disease severity and paralysis. Left graph: Cumulative clinical score of EAE mice. The graph shows the cumulative clinical scores of EAE mice, calculated by summing daily scores and dividing by the number of mice in each treatment group. Each circle symbol represents an individual mouse. *P<0.05 as determined by two-tailed Mann-Whitney test. Right graph: Paralysis in MOG35-55-induced EAE. Clinical scoring over 25 days post-immunization assessed paralysis after AbG or IgG2a therapeutic treatment (as in FIG. 1). Complete hindlimb paralysis occurred when clinical scores >3 in either or both hindlimbs. The percentage of paralyzed mice was calculated based on clinical score and total mice in each group.

FIG. 19 demonstrates antibody-G protection against myelin damage in MOG-EAE Microscopic images of EAE spinal cord sections stained with luxol fast blue, highlighting myelin presence. The extent of demyelination was quantified in spinal cord sections of EAE mice that received therapeutic mu-AbG or IgG2a treatment every 3 days starting from day 3. The data were collected from n=7 mice (mu-AbG) and n=10 mice (IgG2a). Each circle symbol represents an individual mouse. *P<0.05 as determined by two-tailed Mann-Whitney test.

### DESCRIPTION OF THE INVENTION

Provided herein are antibodies (e.g., recombinant and/or monoclonal) that bind one or more epitopes of fibrinogen and fibrin and inhibit inflammatory properties of fibrinogen, without anticoagulant properties.

The practice of the methods and compositions described herein may employ, unless otherwise indicated, conventional techniques of pharmaceutical chemistry, molecular biology, drug formulation techniques, dosage regimes, immunology and biochemistry, all of which are within the skill of those who practice in the art.

### Definitions

For the purposes of clarity and a concise description, features can be described herein as part of the same or separate embodiments; however, it will be appreciated that the scope of the invention may include embodiments having combinations of all or some of the features described.

The terminology used herein is for the purpose of describing particular embodiments only and is not intended to be limiting of the invention. Unless defined otherwise, all technical and scientific terms used herein have the same meaning as commonly understood by one of ordinary skill in the art to which this invention belongs. The following definitions are intended to aid the reader in understanding the present invention but are not intended to vary or otherwise limit the meaning of such terms unless specifically indicated.

As used herein, the indefinite articles “a”, “an” and “the” should be understood to include plural reference unless the context clearly indicates otherwise. Thus, for example, reference to “an inhibitor” refers to one or more agents with the ability to inhibit a target molecule, and reference to “the method” includes reference to equivalent steps and methods known to those skilled in the art, and so forth.

The phrase “and/or,” as used herein, should be understood to mean “either or both” of the elements so conjoined, e.g., elements that are conjunctively present in some cases and disjunctively present in other cases.

As used herein, “of” should be understood to have the same meaning as “and/or” as defined above. For example, when separating a listing of items, “and/or” or “or” shall be interpreted as being inclusive, e.g., the inclusion of at least one, but also including more than one of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “of” as used herein shall only be interpreted as indicating exclusive alternatives (i.e., “one or the other but not both”) when preceded by terms of exclusivity, such as “either,” “one of,” “only one of,” or “exactly one of.”

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

As used herein, the term “about” means plus or minus 10% of the indicated value. For example, about 100 means from 90 to 110. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about.”

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both limits, ranges excluding either both of those included limits are also included in the invention.

Fibrinogen is a soluble precursor to fibrin and both retain the 7C domain.

As used herein, “detecting” refers to the action or process of identifying the presence of that which is being detected, such as fibrin/fibrinogen in a sample.

As used herein, the term “sample” is defined as blood, serum, plasma, urine, saliva, tears, cerebrospinal fluid, supernatant from normal cell lysates, supernatant from pre-neoplastic cell lysates, supernatant from neoplastic cell lysates, supernatants from carcinoma cell lines maintained in tissue culture, and breast aspirates or biopsies. Thus, any number of biological samples can be used in the immunoassays described herein including, without limitation, blood, serum, plasma, urine, saliva, tears, cerebrospinal fluid, supernatant from cell lysates (e.g., normal cells, pre-neoplastic cells, neoplastic cells, carcinoma cells), or breast aspirates or biopsies.

As used herein, “monitoring” refers to the action or process of identifying the presence of that which is being detected at least twice over a period of time.

The term “antibodies” refers to an intact antibody or an antigen-binding portion or fragment thereof that competes with the intact antibody for antigen binding. The term “antibodies” also includes any type of antibody molecule or specific binding molecule that specifically binds to one or more or, such multiple, epitopes of fibrin or fibrinogen. The terms “antigen-binding portion” of an antibody, “antigen-binding fragment” of an antibody, and the like, as used herein, include any naturally occurring, enzymatically obtainable, synthetic, or genetically engineered polypeptide, glycoprotein, or immunoglobulin that specifically binds to one or more epitopes of fibrin or fibrinogen. Antigen-binding fragments of an antibody may be derived, e.g., from full antibody molecules using any suitable standard techniques such as proteolytic digestion or recombinant genetic engineering techniques involving the manipulation and expression of nucleic acids encoding antibody variable and optionally constant domains.

The term “antibody”, also referred to in the art as “immunoglobulin” (Ig), used herein refers to a protein constructed from paired heavy and light polypeptide chains; various Ig isotypes exist, including IgA, IgD, IgE, IgG, and IgM. In one aspect of the antibody provided herein, the antibody comprises the constant region of IgG, IgA, IgD, IgE or IgM, including for example IgG1, IgG2, IgG3 or IgG4, including IgG1k (gamma H chain constant sequence). When an antibody is correctly folded, each chain folds into a number of distinct globular domains joined by more linear polypeptide sequences. For example, the immunoglobulin light chain folds into a variable (VL) and a constant (CL) domain, while the heavy chain folds into a variable (VH) and three constant (CH, CH2, CH3) domains. Interaction of the heavy and light chain variable domains (VH and VL) results in the formation of an antigen binding region (Fv). Each domain has a well-established structure familiar to those of skill in the art.

The light and heavy chain variable regions are responsible for binding the target antigen and can therefore show significant sequence diversity between antibodies. The constant regions show less sequence diversity and are responsible for binding a number of natural proteins to elicit important immunological events. The variable region of an antibody contains the antigen binding determinants of the molecule, and thus determines the specificity of an antibody for its target antigen. The majority of sequence variability occurs in six hypervariable regions, three each per variable heavy and light chain; the hypervariable regions combine to form the antigen-binding site and contribute to binding and recognition of an antigenic determinant. The specificity and affinity of an antibody for its antigen is determined by the structure of the hypervariable regions, as well as their size, shape and chemistry of the surface they present to the antigen. Various schemes exist for identification of the regions of hypervariability, the two most common being those of Kabat and of Chothia and Lesk. Kabat et al (1991a; 1991b) define the “complementarity-determining regions” (CDR) based on sequence variability at the antigen-binding regions of the VH and VL domains. Chothia and Lesk (1987) define the “hypervariable loops” (H or L) based on the location of the structural loop regions in the VH and VL domains. As these individual schemes define CDR and hypervariable loop regions that are adjacent or overlapping, those of skill in the antibody art often utilize the terms “CDR” and “hypervariable loop” interchangeably, and they may be so used herein. For this reason, the regions forming the antigen-binding site are referred to as CDR L1, CDR L2, CDR L3, CDR H1, CDR H2, CDR H3 in the case of antibodies comprising a VH and a VL domain; or as CDR1, CDR2, CDR3 in the case of the antigen-binding regions of either a heavy chain or a light chain. The CDR/loops are referred to herein according to the IMGT numbering system (Lefranc et al., 2003), which was developed to facilitate comparison of variable domains.

An “antibody fragment” as generally referred to herein may include any suitable antigen-binding antibody fragment known in the art. The antibody fragment may be a naturally occurring antibody fragment or may be obtained by manipulation of a naturally occurring antibody or by using recombinant methods. For example, an antibody fragment may include, but is not limited to a Fv, single-chain Fv (scFv; a molecule consisting of VL and VH connected with a peptide linker), Fab, F(ab′)2, single domain antibody (sdAb; a fragment composed of a single VL or VH), and multivalent presentations of any of these.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

Epitope: An antigenic determinant. An epitope is the particular chemical groups or peptide sequences on a molecule that are antigenic, that is, that elicit a specific immune response. An antibody specifically binds a particular antigenic epitope, e.g., on a polypeptide. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, or 8 to 10 amino acids in a unique spatial conformation. Methods of determining spatial conformation of epitopes include, for example, x-ray crystallography and 2-dimensional nuclear magnetic resonance. See, e.g., “Epitope Mapping Protocols” in Methods in Molecular Biology, Vol. 66, Glenn E. Morris, Ed (1996). In one embodiment, an epitope binds an MHC molecule, such an HLA molecule or a DR molecule. These molecules bind polypeptides having the correct anchor amino acids separated by about eight to about ten amino acids, such as nine amino acids.

The term “antigen” or “Ag” as used herein is defined as a molecule that provokes an immune response. This immune response may involve either antibody production, or the activation of specific immunologically competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be synthesized or can be derived from a biological sample. Such a biological sample can include, but is not limited to a tissue sample, a tumor sample, a cell or a biological fluid.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e g, naked or contained in liposomes) and viruses (e.g., lentiviruses, retroviruses, adenoviruses, and adeno-associated viruses) that incorporate the recombinant polynucleotide.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

Any suitable expression vector can be used. For example, prokaryotic cloning vectors include plasmids from E. coli, such as colEl, pCRl, pBR322, pMB9, pUC, pKSM, and RP4. Prokaryotic vectors also include derivatives of phage DNA such as Ml3 and other filamentous single-stranded DNA phages. An example of a vector useful in yeast is the 2μ plasmid. Suitable vectors for expression in mammalian cells include well-known derivatives of SV-40, adenovirus, retrovirus-derived DNA sequences and shuttle vectors derived from combination of functional mammalian vectors, such as those described above, and functional plasmids and phage DNA.

Additional eukaryotic expression vectors are known in the art (e.g., P J. Southern & P. Berg, J. Mol. Appl. Genet, 1:327-341 (1982); Subramani et al, Mol. Cell. Biol, 1: 854-864 (1981); Kaufinann & Sharp, “Amplification And Expression of Sequences Cotransfected with a Modular Dihydrofolate Reductase Complementary DNA Gene,” J. Mol. Biol, 159:601-621 (1982); Kaufhiann & Sharp, Mol. Cell. Biol, 159:601-664 (1982); Scahill et al., “Expression And Characterization Of The Product Of A Human Immune Interferon DNA Gene In Chinese Hamster Ovary Cells,” Proc. Nat'l Acad. Sci USA, 80:4654-4659 (1983); Urlaub & Chasin, Proc. Nat'l Acad. Sci USA, 77:4216-4220, (1980), all of which are incorporated by reference herein).

The expression vectors useful in the present invention contain at least one expression control sequence that is operatively linked to the DNA sequence or fragment to be expressed. The control sequence is inserted in the vector in order to control and to regulate the expression of the cloned DNA sequence. Examples of useful expression control sequences are the lac system, the trp system, the tac system, the trc system, major operator and promoter regions of phage lambda, the control region of fd coat protein, the glycolytic promoters of yeast, e.g., the promoter for 3-phosphoglycerate kinase, the promoters of yeast acid phosphatase, e.g., Pho5, the promoters of the yeast alpha-mating factors, and promoters derived from polyoma, adenovirus, retrovirus, and simian virus, e.g., the early and late promoters or SV40, and other sequences known to control the expression of genes of prokaryotic or eukaryotic cells and their viruses or combinations thereof.

The present invention also provides recombinant host cells containing the expression vectors previously described.

Cell lines of can be selected based on high level of expression, constitutive expression of protein of interest and minimal contamination from host proteins. Mammalian cell lines available as hosts for expression are well known in the art and include many immortalized cell lines, such as but not limited to, Chinese Hamster Ovary (CHO) cells, Baby Hamster Kidney (BHK) cells and many others. Suitable additional eukaryotic cells include yeast and other fungi. Useful prokaryotic hosts include, for example, E. coli, such as E. coli SG-936, E. coli HB 101, E. coli W3110, E. coli X1776, E. coli X2282, E. coli DHI, and E. coli MRC1, Pseudomonas, Bacillus, such as Bacillus subtilis, and Streptomyces.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

“Variants” are biologically active antibodies or fragments thereof having an amino acid sequence that differs from the sequences provided herein, by virtue of an insertion, deletion, modification and/or substitution of one or more amino acid residues within the comparative sequence. Variants generally have less than 100% sequence identity with the comparative sequence. Ordinarily, however, a biologically active variant will have an amino acid sequence with at least about 70% amino acid sequence identity with the comparative sequence, such as at least about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity. The variants include peptide fragments of at least 10 amino acids that retain binding ability. Variants also include polypeptides wherein one or more amino acid residues are added at the N- or C-terminus of, or within, the comparative sequence. Variants also include polypeptides where a number of amino acid residues are deleted and optionally substituted by one or more amino acid residues. Variants also may be covalently modified, for example by substitution with a moiety other than a naturally occurring amino acid or by modifying an amino acid residue to produce a non-naturally occurring amino acid.

“Percent amino acid sequence identity” is defined herein as the percentage of amino acid residues in the candidate sequence that are identical with the residues in the sequence of interest, such as the polypeptides of the invention, after aligning the sequences and introducing gaps, if necessary, to achieve the maximum percent sequence identity, and not considering any conservative substitutions as part of the sequence identity. None of N-terminal, C-terminal, or internal extensions, deletions or insertions into the candidate sequence shall be construed as affecting sequence identity or homology. Methods and computer programs for the alignment are well known in the art, such as “BLAST”.

A substantially identical sequence may comprise one or more conservative amino acid mutations. It is known in the art that one or more conservative amino acid mutations to a reference sequence may yield a mutant peptide with no substantial change in physiological, chemical, or functional properties compared to the reference sequence; in such a case, the reference and mutant sequences would be considered “substantially identical” polypeptides. Conservative amino acid mutation may include addition, deletion, or substitution of an amino acid; a conservative amino acid substitution is defined herein as the substitution of an amino acid residue for another amino acid residue with similar chemical properties (e.g. size, charge, or polarity).

In a non-limiting example, a conservative mutation may be an amino acid substitution. Such a conservative amino acid substitution may substitute a basic, neutral, hydrophobic, or acidic amino acid for another of the same group. By the term “basic amino acid” it is meant hydrophilic amino acids having a side chain pK value of greater than 7, which are typically positively charged at physiological pH. Basic amino acids include histidine (His or H), arginine (Arg or R), and lysine (Lys or K). By the term “neutral amino acid” (also “polar amino acid”), it is meant hydrophilic amino acids having a side chain that is uncharged at physiological pH, but which has at least one bond in which the pair of electrons shared in common by two atoms is held more closely by one of the atoms. Polar amino acids include serine (Ser or S), threonine (Thr or T), cysteine (Cys or C), tyrosine (Tyr or Y), asparagine (Asn or N), and glutamine (Gln or Q). The term “hydrophobic amino acid” (also “non-polar amino acid”) is meant to include amino acids exhibiting a hydrophobicity of greater than zero according to the normalized consensus hydrophobicity scale of Eisenberg (1984). Hydrophobic amino acids include proline (Pro or P), isoleucine (Ile or I), phenylalanine (Phe or F), valine (Val or V), leucine (Leu or L), tryptophan (Trp or W), methionine (Met or M), alanine (Ala or A), and glycine (Gly or G). “Acidic amino acid” refers to hydrophilic amino acids having a side chain pK value of less than 7, which are typically negatively charged at physiological pH. Acidic amino acids include glutamate (Glu or E), and aspartate (Asp or D).

Sequence identity is used to evaluate the similarity of two sequences; it is determined by calculating the percent of residues that are the same when the two sequences are aligned for maximum correspondence between residue positions. Any known method may be used to calculate sequence identity; for example, computer software is available to calculate sequence identity. Without wishing to be limiting, sequence identity can be calculated by software such as NCBI BLAST2 service maintained by the Swiss Institute of Bioinformatics (and as found at ca.expasy.org/tools/blast/), BLAST-P, Blast-N, or FASTA-N, or any other appropriate software that is known in the art.

The substantially identical sequences of the present invention may be at least 85% identical; in another example, the substantially identical sequences may be at least 70, 75, 80, 85, 90, 95, 96, 97, 98, 99, or 100% (or any percentage there between) identical at the amino acid level to sequences described herein. In specific aspects, the substantially identical sequences retain the activity and specificity of the reference sequence. In a non-limiting embodiment, the difference in sequence identity may be due to conservative amino acid mutation(s).

The terms “subject,” “mammal” and “mammalian subject” as used herein refers to any animal classified as a mammal, including humans, higher non-human primates, rodents, and domestic and farm animals, such as cows, horses, dogs, and cats. In some embodiments of the invention, the mammal is a human (male or female). The term “subject” as used herein refers to any member of the animal kingdom, typically a mammal. The term “mammal” refers to any animal classified as a mammal, including humans, other higher primates, domestic and farm animals, and zoo, sports, or pet animals, such as dogs, cats, cattle, horses, sheep, pigs, goats, rabbits, etc. Typically, the mammal is human.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

As used herein, the terms “treat,” “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in an animal, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causing regression of the disease, e.g., to completely or partially remove symptoms of the disease.

The term “prevent,” as used herein, means to stop something from happening, or taking advance measures against something possible or probable from happening. In the context of medicine, “prevention” generally refers to action taken to decrease the chance of getting a disease or condition.

Administration “in combination with” one or more further therapeutic agents includes simultaneous (concurrent) and consecutive administration in any order.

“Parenteral” administration of an immunogenic composition includes, e.g., subcutaneous (s.c.), intravenous (i.v.), intramuscular (i.m.), or intrasternal injection, or infusion techniques.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

As used herein, an “effective amount” means an amount sufficient to produce a selected effect, such as alleviating symptoms of a disease or disorder. In the context of administering two or more compounds, the amount of each compound, when administered in combination with another compound(s), may be different from when that compound is administered alone. The term “more effective” means that the selected effect is alleviated to a greater extent by one treatment relative to the second treatment to which it is being compared.

Moreover, a treatment regime of a subject with a therapeutically effective amount may consist of a single administration, or alternatively comprise a series of applications. The length of the treatment period depends on a variety of factors, such as the severity of the disease, the age of the subject, the concentration of the agent, the responsiveness of the patient to the agent, or a combination thereof. It will also be appreciated that the effective dosage of the agent used for the treatment may increase or decrease over the course of a particular treatment regime. Changes in dosage may result and become apparent by standard diagnostic assays known in the art. The antibodies described herein may, in aspects, be administered before, during or after treatment with conventional therapies for the disease or disorder in question, such as MS.

The antibodies described herein can be formulated in a unit dosage such as a solution, suspension or emulsion, in association with a carrier. Such carriers are inherently nontoxic and nontherapeutic. As used herein, carrier is intended to include any and all solvents, dispersion media, coatings, antibacterial and antifungal agents, isotonic and absorption delaying agents, and the like, compatible with administration. Suitable carriers are described in the most recent edition of Remington's Pharmaceutical Sciences, a standard reference text in the field, which is incorporated herein by reference. Examples of such carriers or diluents include, but are not limited to, water, saline, Ringer's solutions, dextrose solution, and 5% human serum albumin. The carrier may contain minor amounts of additives such as substances that enhance chemical stability, including buffers and preservatives.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

A composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration.

The compositions for administration can include a solution of the antibody dissolved in a pharmaceutically acceptable carrier, such as an aqueous carrier. A variety of aqueous carriers can be used, for example, buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of antibody in these formulations can vary widely, and will be selected primarily based on fluid volumes, viscosities, body weight and the like in accordance with the particular mode of administration selected and the subject's needs.

A typical pharmaceutical composition for intravenous administration includes about 0.1 to 10 mg of antibody per subject per day, such as 1 mg/kg of subject/patient weight per day. Dosages from 0.1 up to about 100 mg per subject per day may be used. Actual methods for preparing administrable compositions will be known or apparent to those skilled in the art and are described in more detail in such publications as Remington's Pharmaceutical Science, 19th ed., Mack Publishing Company, Easton, Pa. (1995).

Antibodies may be provided in lyophilized form and rehydrated with sterile water before administration, although they are also provided in sterile solutions of known concentration. The antibody solution is then added to an infusion bag containing 0.9% sodium chloride, USP, and in some cases administered at a dosage of from 0.5 to 15 mg/kg of body weight. Antibodies can be administered by slow infusion, rather than in an intravenous push or bolus. In one example, a higher loading dose is administered, with subsequent, maintenance doses being administered at a lower level. For example, an initial loading dose of 4 mg/kg may be infused over a period of some 90 minutes, followed by weekly maintenance doses for 4-8 weeks of 2 mg/kg infused over a 30-minute period if the previous dose was well tolerated.

Administration of the antibodies, fusion proteins and immunoconjugates (or compositions thereof) disclosed herein can also be accompanied by administration of other therapeutic treatments.

As used herein, the terms “including”, “includes”, “having”, “has”, “with”, or variants thereof, are intended to be inclusive similar to the term “comprising.”

As used herein, said “contain”, “have” or “including” include “comprising”, “mainly consist of”, “basically consist of” and “formed of”; “primarily consist of”, “generally consist of” and “comprising of” belong to generic concept of “have” “include” or “contain”.

The terms “comprises,” “comprising,” and the like can have the meaning ascribed to them in U.S. Patent Law and can mean “includes,” “including” and the like. As used herein, “including” or “includes” or the like means including, without limitation.

It will be understood that any aspects described as “comprising” certain components may also “consist of” or “consist essentially of,” wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known pharmaceutically acceptable additive, excipient, diluent, carrier, and the like. Typically, a composition consisting essentially of a set of components will comprise less than 5% by weight, typically less than 3% by weight, more typically less than 1% by weight of non-specified components.

Methods involving conventional molecular biology techniques are described herein. Such techniques are generally known in the art and are described in detail in methodology treatises, such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22: 1859-1862, 1981, and Matteucci et al., J. Am. Chem. Soc. 103:3185, 1981.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### Antibodies/Polypeptides

Antibodies from multiple sclerosis (MS) patients' cerebrospinal fluid (CSF) plasma cells were produced. The antibodies were screened to identify target antigens, such as by immunoprecipitation followed by mass spectrometry. Eighteen antibodies with fibrinogen as a potential target antigen were identified. These antibodies were further screened for their binding potential. One antibody (referred to herein as antibody G or 45-C1C3) was determined to bind fibrinogen and fibrin, bind the fibrin epitope P2, and inhibit inflammatory properties of fibrinogen, without anticoagulant properties.

Antibody 45-C1C3 is a human IgGk resulting from a single CSF plasma cell obtained from a human subject with a MS diagnosis. 45-C1C3-VH and 45-C1C3-VLk PCR products were separately cloned into eukaryotic expression plasmid containing the constant regions of human IgG1 H chain, and human kL chain as described in Tiller et al., 2008 (J of Immunological Methods 329 (2008) 112-124). After cloning and confirmation of cloned 45-C1C3-VH and 45-C1C3-VLk sequences, plasmid DNA was produced in E. coli and purified (sequences provided herein). Soluble 45-C1C3 antibody was produced by transient expression in 293-T human embryonic kidney fibroblasts following co-transfection with 45-C1C3-VH and 45-C1C3-VLk encoding plasmid DNA by calcium phosphate precipitation. Transfected cells were cultured in serum and Ig free D-MEM. Supernatants were collected after 8 days of culture and 45-C1C3 antibody was purified using protein G sepharose.

A light or heavy chain variable region of an antibody has four framework regions interrupted by three hypervariable regions, known as complementary determining regions (CDRs). CDRs determine the specificity of antigen binding. The heavy chain and light chain each have three CDRs, designated from the N terminus as CDR1, CDR2, and CDR3 with the four framework regions flanking these CDRs. The amino acid sequences of the framework region are highly conserved and CDRs can be transplanted into other antibodies. Therefore, a recombinant antibody can be produced by combining CDRs from one or more antibodies with the framework of one or more other antibodies.

Polypeptides/antibodies of the invention comprise full-length heavy chain variable regions, full-length light chain variable regions, binding fragments or variants thereof, and combinations thereof.

G Heavy Chain Variable Region (V-D-J)
Nucleotide Sequence:
(SEQ ID NO: 2; cDNA)
gaagtgcagctggtggagtctgggggaggcgcggtccaacctgggaggtc
cctgagactctcctgtgcagcctctggagtcagtttcagtaacattggca
tgcactgggtccgccaggctccaggcaaggggctggagtgggtggcactt
atatcatctgatggacgtcatacacactatgcagactccgtgaagggccg
attcaccatctccagagacaattccgagaacacgctctatctacagatca
acggcctgagagctgacgacacggctgtttattactgtgcgaaaggcctt
gacactcgtgcccgttacatggggaaattttatacttttgactactgggg
ccagggaaccctggtcaccgtctcctca
Amino acid Sequence:
(SEQ ID NO: 3)
EVQLVESGGGAVQPGRSLRLSCAASGVSFSNIGMHWVRQAPGKGLEWVAL
ISSDGRHTHYADSVKGRFTISRDNSENTLYLQINGLRADDTAVYYCAKGL
DTRARYMGKFYTFDYWGQGTLVTVSS
G Kappa Light Chain Variable Region (V-J)
Nucleotide Sequence:
(SEQ ID NO: 4; cDNA)
gacatccagttgacccagtctccatcctccctgtctgcatctgtaggaga
cagagtcaccatcacttgtcgggcaagtcagaacattggcgtcaatttaa
attggtatcaacagagaccagggaaaggcccgaaactcctagtctcttct
acatttagtttgcaaagtggggtcccatcaaggtttagtgccagtggagc
tgggacaaatttcactctcaccatcagcagtctgcaacctgaggactatg
tcacttactactgtcaacagagttacagtagtccattcacattcggccct
gggaccaaagtggatatcaaa
Amino acid Sequence:
(SEQ ID NO: 5)
DIQLTQSPSSLSASVGDRVTITCRASQNIGVNLNWYQQRPGKGPKLLVSS
TFSLQSGVPSRESASGAGTNFTLTISSLQPEDYVTYYCQQSYSSPFTFGP
GTKVDIK

A polypeptide or nucleotide variant, antibody variant or variant heavy or light chain differs by about, for example, 1, 2, 3, 4, 5, 6, 7, 8, 9, 10, 20, 30, 40, 50, 60 or more amino acids or nucleotides (e.g., amino acid additions, substitutions or deletions) from a polypeptide or nucleic acid sequence shown in SEQ ID NOs: 2-5 or a fragment thereof. Where this comparison requires alignment, the sequences are aligned for maximum homology. The site of variation can occur anywhere in the polypeptide or nucleic acid sequence. In one embodiment of the invention a variant polypeptide has activity substantially similar to a polypeptide shown in SEQ ID NOs: 3 or 5. Activity substantially similar means that when the polypeptide is used to construct an antibody, the antibody has the same or substantially the same activity/binding to fibrin/or fibrinogen.

As used herein, percent identity of two amino acid sequences (or of two nucleic acid sequences) is determined using the algorithm of Karlin and Altschul (PNAS USA 87:2264-2268, 1990), modified as in Karlin and Altschul, PNAS USA 90:5873-5877, 1993). Such an algorithm is incorporated into the NBLAST and XBLAST programs of Altschul et al. (J. Mol. Biol. 215:403-410, 1990). BLAST nucleotide searches are performed with the NBLAST program, score=100, wordlength=12. BLAST protein searches are performed with the XBLAST program, score=50, wordlength=3. To obtain gapped alignment for comparison purposes GappedBLAST is utilized as described in Altschul et al. (Nucleic Acids Res. 25:3389-3402, 1997). When utilizing BLAST and GappedBLAST programs the default parameters of the respective programs (e.g., XBLAST and NBLAST) are used to obtain nucleotide sequences homologous to a nucleic acid molecule of the invention.

Identity or identical means amino acid sequence (or nucleic acid sequence) similarity and has an art recognized meaning. Sequences with identity share identical or similar amino acids (or nucleic acids). Sequence identity is the percentage of amino acids identical to those in the antibody's original amino acid sequence, determined after the sequences are aligned and gaps are appropriately introduced to maximize the sequence identity as necessary. Thus, a candidate sequence sharing 85% amino acid sequence identity with a reference sequence requires that, following alignment of the candidate sequence with the reference sequence, 85% of the amino acids in the candidate sequence are identical to the corresponding amino acids in the reference sequence, and/or constitute conservative amino acid changes. The sequences provided herein can include 80%, 85%, 90%, 95%, 97%, 98%, 99% or 100% identity to SEQ ID NOs:2-5.

Methods of introducing a mutation into an amino acid sequence are well known to those skilled in the art. See, e.g., Ausubel (ed.), Current Protocols in Molecular Biology, John Wiley and Sons, Inc. (1994); Maniatis et al., Molecular Cloning: A Laboratory Manual, Cold Spring Harbor laboratory, Cold Spring Harbor, N.Y. (1989)). Mutations can also be introduced using commercially available kits such as “QuikChange™ Site-Directed Mutagenesis Kit” (Stratagene). The generation of a functionally active variant polypeptide by replacing an amino acid that does not influence the function of a polypeptide can be accomplished by one skilled in the art.

The variant polypeptides can have conservative amino acid substitutions at one or more predicted non-essential amino acid residues. A conservative substitution is one in which an amino acid is substituted for another amino acid that has similar properties, such that one skilled in the art of peptide chemistry would expect the secondary structure and hydropathic nature of the polypeptide to be substantially unchanged. In general, the following groups of amino acids represent conservative changes: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his.

A polypeptide or antibody of the invention can be covalently or non-covalently linked to an amino acid sequence to which the polypeptide or antibody is not normally associated with in nature. Additionally, a polypeptide or antibody of the invention can be covalently or non-covalently linked to compounds or molecules other than amino acids. For example, a polypeptide or antibody can be linked to an indicator reagent (indicator reagents can include chromogenic agents, catalysts, such as enzyme conjugates, fluorescent compounds, such as fluorescein and rhodamine, chemiluminescent compounds, such as dioxetanes, acridiniums, phenanthridiniums, ruthenium, and luminol, radioactive elements, direct visual labels, as well as cofactors, inhibitors, magnetic particles, and the like; examples of enzyme conjugates include alkaline phosphatase, horseradish peroxidase, beta-galactosidase, and the like), an amino acid spacer, an amino acid linker, a signal sequence, a stop transfer sequence, a transmembrane domain, a protein purification ligand (e.g., glutathione-S-transferase, histidine tag, and staphylococcal protein A), or a combination thereof. In one embodiment of the invention a protein purification ligand can be one or more C amino acid residues at, for example, the amino terminus or carboxy terminus of a polypeptide of the invention. An amino acid spacer is a sequence of amino acids that are not usually associated with a polypeptide or antibody of the invention in nature. An amino acid spacer can comprise about 1, 5, 10, 20, 100, or 1,000 amino acids. Conjugated antibodies can be bound to various molecules including, for example, polymers, hyaluronic acid, fluorescent substances, luminescent substances, haptens, enzymes, metal chelates, cytotoxic agents, radionuclides, and drugs.

A polypeptide of the invention can be isolated from cells or tissue sources using standard protein purification techniques. Polypeptides of the invention can also be synthesized chemically or produced by recombinant DNA techniques. For example, a polypeptide of the invention can be synthesized using conventional peptide synthesizers.

A polypeptide of the invention can be produced recombinantly. A polynucleotide encoding a polypeptide of the invention can be introduced into a recombinant expression vector, which can be expressed in a suitable expression host cell system using techniques well known in the art. A variety of bacterial, yeast, plant, mammalian, and insect expression systems are available in the art and any such expression system can be used. Optionally, a polynucleotide encoding a polypeptide can be translated in a cell-free translation system.

Antibodies of the invention can be produced using methods available to those of skill in the art.

### Methods of Use

The antibodies disclosed herein can used to treat or prevent neurological/neurodegenerative diseases and disorders including, but not limited to, multiple sclerosis (MS), progressive multifocal leukoencephalopathy (PML), encephalomyelitis (EPL), central pontine myelolysis (CPM), adrenoleukodystrophy, Alexander's disease, Pelizaeus Merzbacher disease (PMZ), Wallerian Degeneration, optic neuritis, transverse myelitis, amyotrophic lateral sclerosis (ALS), Huntington's disease, Alzheimer's disease, Parkinson's disease, spinal cord injury, traumatic brain injury, post radiation injury, neurologic complications of chemotherapy, stroke, acute ischemic optic neuropathy, vitamin E deficiency, isolated vitamin E deficiency syndrome, AR, Bassen-Kornzweig syndrome, Marchiafava-Bignami syndrome, metachromatic leukodystrophy, trigeminal neuralgia, acute disseminated encephalitis, seizures, Guillain-Barre syndrome, Marie-Charcot-Tooth disease and Bell's palsy.

The antibodies disclosed herein also find use in treating any condition/disease in which fibrin plays a role including, but not limited to, inflammation, periodontal disease, rheumatoid arthritis (RA) colitis, cancer (e.g., B cell lymphoma) or muscular dystrophy.

The antibodies disclosed herein also find use in treating effects of coronavirus (e.g., SARS-CoV-2 and/or SARS-CoV-1) infection, including Long COVID or Post-Acute Sequelae of COVID-19 (PASC) that can involve multiple organs including the lung, heart, brain, and joints. Most people who become infected with SARS-CoV-2 (CoVID-19) recover completely within a few weeks. But some people—even those who had mild versions of the disease—continue to experience symptoms after their initial recovery. These people sometimes describe themselves as “long haulers” and the condition has been called post-CoVID-19 syndrome or “long CoVID-19.” As used herein, the long-term adverse effects of SARS-CoV-2 infection occur after about 1-3, or 2 weeks after an initial SARS-CoV-2 infection. In some cases, the SARS-CoV-2 may be detected in these “long haulers” but in other cases the long-term symptoms of SARS-CoV-2 infection occur even when the SARS-CoV-2 virus is no longer detectable.

The anti-fibrin antibodies provided herein can effectively inhibit these adverse physiological responses and symptoms of SARS-CoV-2 infection. In some cases, anti-fibrin antibodies can inhibit the adverse symptoms of SARS-CoV-1 infections.

### Method to Detect

Detection of antibodies similar to 45-C1C3 or other anti-fibrin antibodies in the plasma, serum, or CSF can be used as biomarkers for detecting disease activity in MS patients' and other neurodegenerative/neurological diseases. Also, detection of antibodies similar to 45-C1C3 or other anti-fibrin antibodies can facilitate patient stratification for anti-fibrin therapies in neurologic diseases.

One embodiment of the invention provides methods of detecting fibrin in a sample. The methods comprise contacting the sample suspected of containing fibrin with an antibody or antigen binding portion thereof of the invention to form fibrin/antibody complexes. The presence of the fibrin/antibody complexes are detected, thereby detecting the presence of the fibrin.

The test sample can be, e.g., lymph node or tissue aspirate, serum, whole blood, plasma, circulating tumor cells, tumor cells or tissue (e.g., tissue biopsy) or ascites fluid. Polypeptide/antibody complexes can be detected by any method known in the art, including, but not limited to, enzyme-linked immunosorbent assay (ELISA), multiplex fluorescent immunoassay (MFI or MFIA), radioimmunoassay (RIA), sandwich assay, western blotting, immunoblotting analysis, an immunohistochemistry method, immunofluorescence assay, fluorescence-activated cell sorting (FACS) or a combination thereof.

An immunoassay for fibrin can utilize one antibody or several different antibodies. Immunoassay protocols can be based upon, for example, competition, direct reaction, or sandwich type assays using, for example, labeled antibody. Antibodies of the invention can be labeled with any type of label known in the art, including, for example, fluorescent, chemiluminescent, radioactive, enzyme, colloidal metal, radioisotope and bioluminescent labels.

Antibodies of the invention or antigen-binding portions thereof can be bound to a support and used to detect the presence of fibrin. Supports include, for example, glass, polystyrene, polypropylene, polyethylene, dextran, nylon, amylases, natural and modified celluloses, polyacrylamides, agaroses and magletite.

Antibodies can be used as imaging agents conjugated with radioligands for the detection of fibrin in tissues. Diagnostic agents for use in the method may be radioisotope, a paramagnetic label, a fluorophore, a Near Infra-Red (NIR) fluorochrome or dye, an affinity label, or a detectable protein-based molecule via genetic fusion to the antibody. Detection may be accomplished by any suitable imaging method including, but not limited to non-invasive optical imaging, ultrasound, MRI, PET, or SPECT.

The following are provided for exemplification purposes only and are not intended to limit the scope of the invention described in broad terms above.

### EXAMPLES

### Example I

### Materials and Methods/Results

Generation of Antibodies from MS Patients Including 45-C1C3

### Sample

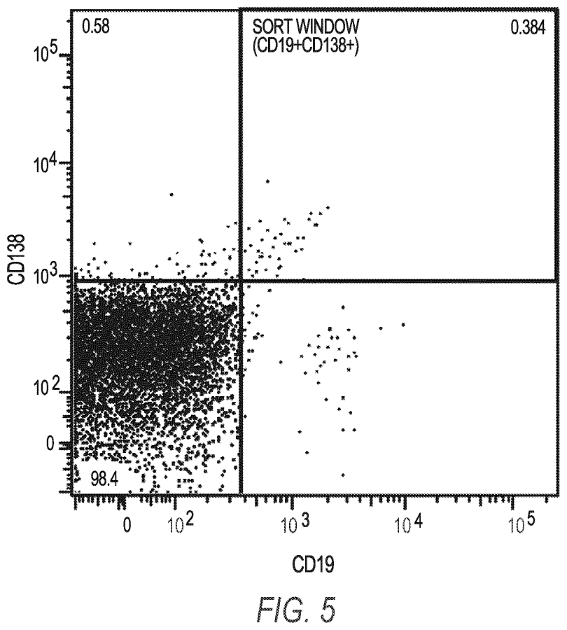

Cerebrospinal fluid (CSF) was obtained from a MS patient after obtaining informed consent and under a UCSF Committee on Human Research (CHR) approved protocol (#10-02389). Immediately after collection, CSF cells were pelleted, resuspended in FACS buffer and incubated with fluorochrome-labeled anti-CD19 and anti-CD138 antibodies. Single CD138+ plasma cells were sorted on a FACS-Aria (Becton Dickinson) into individual wells of multi-well PCR plates (FIG. 5).

### Single Cell RT-PCR of Immunoglobulin Variable Region Heavy and Light Chain Transcripts

Single cell RT-PCR (SC-PCR) of immunoglobulin (Ig) variable region (V) heavy (H) and light (L) chain transcripts was performed using reverse primers annealing to the constant regions of immunoglobulin genes (IgG-heavy chain, XL chain, kL chain) and forward primers specific for the conserved framework region 1 complementary for H or L chain Ig variable region families were used, as described in Tiller et al., 2008. Resulting IgG-VH, Igk-VL, and/or Igλ\-VL were sequenced using standard Sanger sequencing.

### 45-C1C3 Cloning, Production and Purification

Antibody 45-C1C3 is a human IgGk resulting from a single CSF plasma cell obtained from a human subject with a MS diagnosis; 45-C1C3-VH and 45-C1C3-VLk PCR products were separately cloned into eukaryotic expression plasmid containing the constant regions of human IgG1 H chain, and human kL chain as described in Tiller et al., 2008 (which is incorporated herein by reference in its entirety for producing/generating recombinant monoclonal antibodies). During the PCR and cloning process of the variable and/or H and L chains, primers are used that contain restriction sites (for example, restriction sites recognized by restriction enzymes AgeI, SalI, BsiWI; primers are published in Tiller et al. 2008, which is incorporated in its entirety by reference). These restriction sites are not present in natural H and L chain nucleotide sequences, and in the cloning process and represent an entirely non-natural component of antibodies formed therefrom. After cloning and confirmation of cloned 45-C1C3-VH and 45-C1C3-VLk sequences, plasmid DNA was produced in E. coli and purified (sequences provided herein). Soluble 45-C1C3 antibody was produced by transient expression in 293-T human embryonic kidney fibroblasts following co-transfection with 45-C1C3-VH and 45-C1C3-VLk encoding plasmid DNA by calcium phosphate precipitation. Transfected cells were cultured in serum and Ig free D-MEM. Supernatants were collected after 8 days of culture and 45-C1C3 antibody was purified using protein G sepharose.

Identification of Fibrinogen as Potential Target Antigen of Human Antibodies Derived from MS Patients, Including 45-C1C3

Antibody 45-C1C3 and other monoclonal antibodies were used in unbiased immunoprecipitation/mass-spectrometry experiments to determine their target antigens. Immunoprecipitation reactions were conducted using monoclonal antibodies (including 45-C1C3) and human brain myelin homogenate (isolated from human MS brain tissue). First, individual monoclonal antibodies were added to Protein A/G coated multi-well plates; subsequently, myelin homogenate was added to each individual well. Following repeated washes, monoclonal antibodies and captured target antigens were eluted from the protein A/G plates.

Eluates including monoclonal antibody and captured target antigens were prepared for mass spectrometry using in-solution digestion method. Mass spectrometry results suggested fibrinogen as potential target antigen of a number of monoclonal antibodies, including 45-C1C3. Eighteen antibodies, including 45-C1C3, other potential fibrinogen-binding antibodies, and antibodies that did capture fibrinogen, were investigated in a blinded fashion; blinded antibodies were labeled “A” thru “R”.

Identification of 45-C1C3 Antibody Derived from MS Patients Binds to Fibrinogen and Fibrin

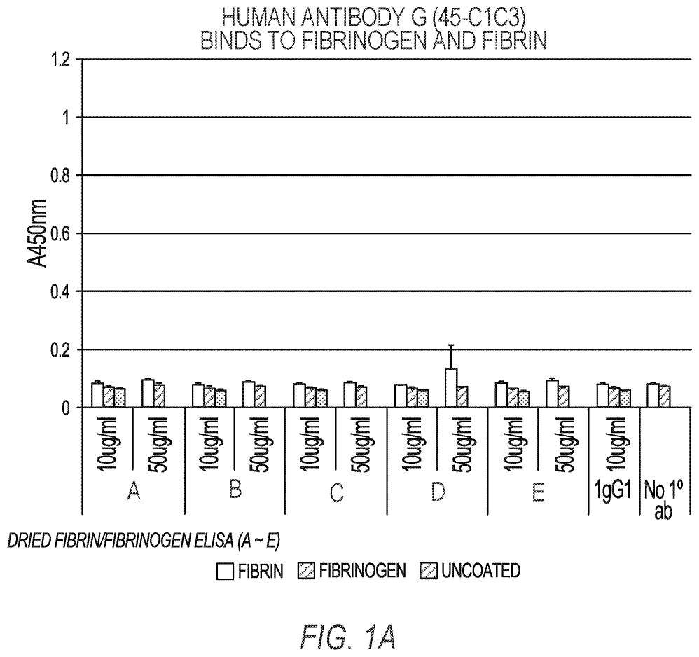

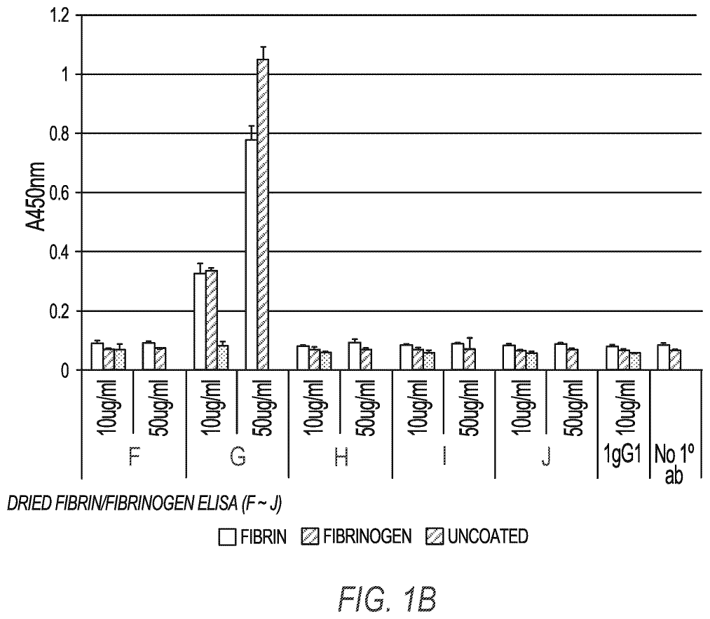

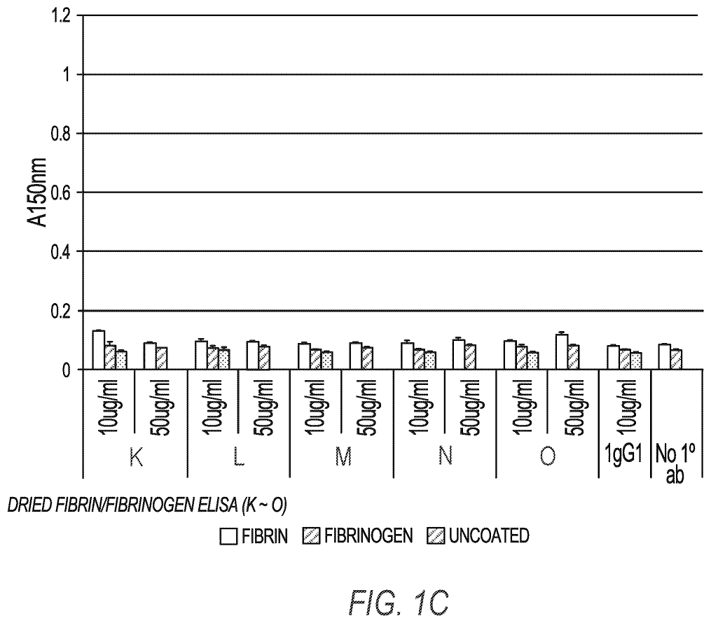

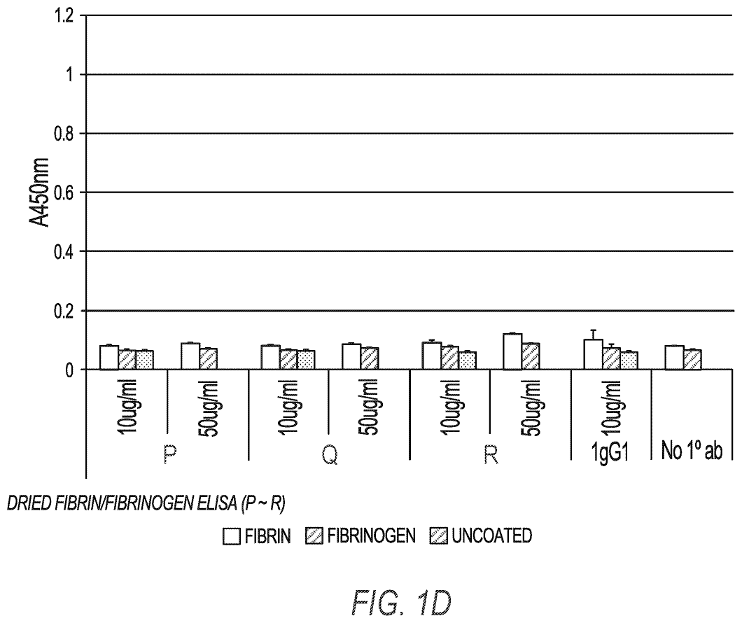

A screen was performed in an ELISA-based assay in 96-well plates coated with fibrinogen or fibrin to determine if the antibodies with potential binding to fibrinogen based on the mass-spec results recognized fibrinogen or fibrin. Fibrinogen and fibrin networks were left to air dry overnight at 37° C. Only one antibody, designated mAb G (45-C1C3), was found to bind to both fibrin and fibrinogen in a dose-dependent manner (FIGS. 1A-1D). The remaining antibodies, including some with potential binding to fibrinogen from the mass-spec analysis, did not show binding to fibrin and fibrinogen at the concentrations tested.

Identification that 45-C1C3 does not have Anti-Coagulant Activity.

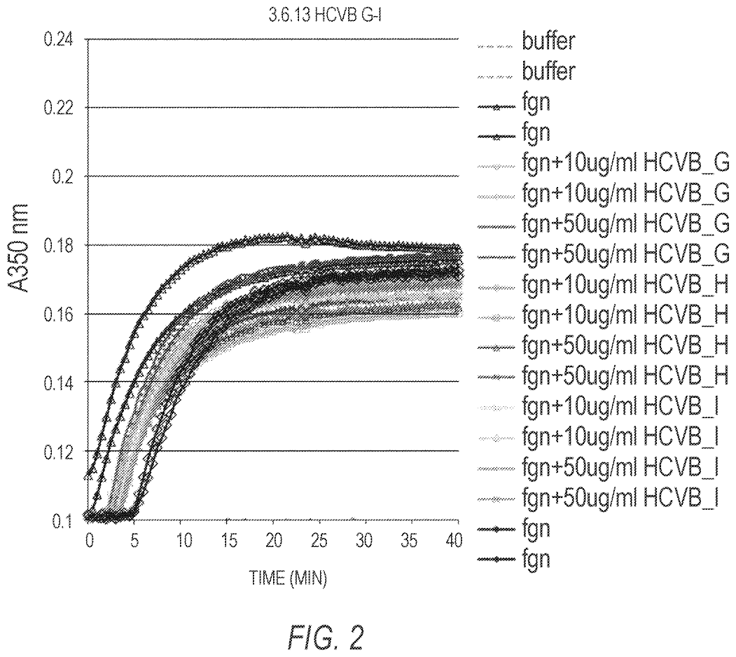

Fibrinogen regulates blood coagulation by engaging the platelet α_{IIb}β₃ integrin receptor via its γ⁴⁰⁸⁻⁴¹¹ epitope, and it mediates inflammatory processes by engaging the Mac-1 receptor via its γ³⁷⁷⁻³⁹⁵ epitope. As a result, fibrinogen knock-in mice, in which the γ³⁹⁰⁻³⁹⁶ Mac-1 binding site has been mutated, show normal coagulation properties, such as platelet aggregation, thrombus formation, and clotting time. To determine if the human mAbs interfered with blood coagulation, their effects on the procoagulant properties of fibrinogen in vitro were examined first (FIG. 2). Antibody G (45-C1C3) did not inhibit fibrin polymerization, despite binding to fibrin and fibrinogen (FIG. 2).

Identification of 45-C1C3 as an antibody derived from MS patients that inhibits fibrin-induced inflammatory gene expression in macrophages.

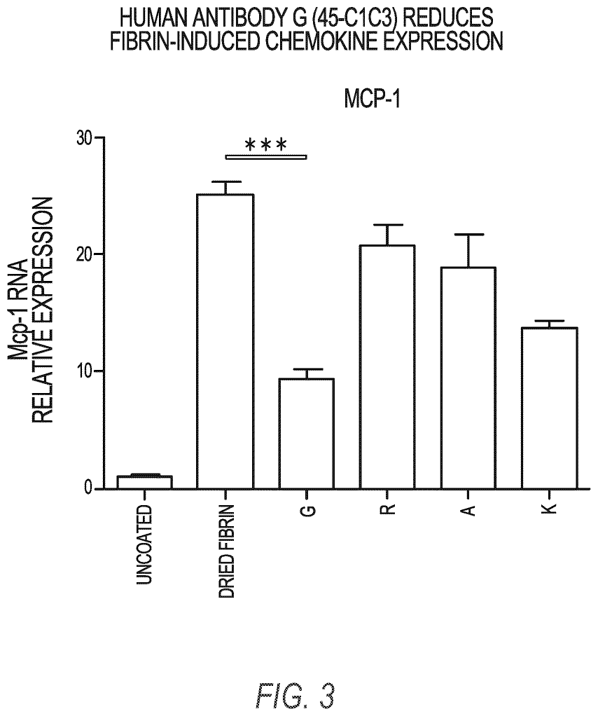

Chemokine expression is a major function of activated microglia and macrophages that is mediated by CD11b/CD18. To determine the functional potency of human mAbs targeting the γ³⁷⁷⁻³⁹⁵ epitope, a chemokine gene expression assay was performed on cultured bone marrow derived macrophages (BMDMs) in fibrin-coated plates. mAbs A and G, bind P2 without altering fibrinogen polymerization. As a control, antibody R was selected, in which mild anti-coagulation activity was detected. mAb K was used as a further control. Tissue culture wells were coated with 100 g/ml fibrin upon which BMDMs (100,000 cells/well) were plated in the presence of these mAbs. Real time-PCR analysis of chemokine Mcp-1 expression in BMDMs was performed at 6 h in the presence and absence of mAbs (FIG. 3). Expression of Mcp-1 was decreased with mAb G but not with the other mAbs. Overall, mAb G (45-C1C3) was confirmed to have reactivity with only the targeted fibrinogen epitope (γ³⁷⁷⁻³⁹⁵) and showed superior efficacy in inhibiting chemokine expression in vitro.

### Discussion

Screening of human antibodies derived from MS patients identified human antibody G (45-C1C3) as a unique antibody with properties to bind fibrinogen and fibrin and inhibit proinflammatory properties of fibrin in macrophages without affecting blood coagulation. 45-C1C3 is the first identified human antibody that shares preferential binding to fibrin than fibrinogen and the functional properties of the mouse anti-P2 antibody 5B8. The antibody can have a variety of therapeutic implications in human disease by selectively suppressing innate immunity within and outside of the nervous system. The antibody may also accelerate fibrin removal from the CNS via increasing phagocytosis. Detection of antibodies similar to 45-C1C3 or other anti-fibrin antibodies in the plasma, serum, or CSF can be used as biomarkers for detecting disease activity in MS patients' and other neurodegenerative or neurological diseases. Also, detection of antibodies similar to 45-C1C3 or other anti-fibrin antibodies can facilitate patient stratification for anti-fibrin therapies in neurologic diseases.

### BIBLIOGRAPHY

Adams, R. A., J. Bauer, M. J. Flick, S. L. Sikorski, T. Nuriel, H. Lassmann, J. L. Degen, and K. Akassoglou. 2007a. The fibrin-derived gamma377-395 peptide inhibits microglia activation and suppresses relapsing paralysis in central nervous system autoimmune disease. J Exp Med 204:571-582.
Adams, R. A., C. Schachtrup, D. Davalos, I. Tsigelny, and K. Akassoglou. 2007b. Fibrinogensignal transduction as a mediator and therapeutic target in inflammation: Lessons from Multiple Sclerosis. Curr Med Chem 14:2925-2936.
Davalos, D., and K. Akassoglou. 2012. Fibrinogen as a key regulator of inflammation in disease. Semin Immunopathol 34:43-62.
Davalos, D., J. K. Ryu, M. Merlini, K. M. Baeten, N. Le Moan, M. A. Petersen, T. J.

Deerinck, D. S. Smirnoff, C. Bedard, H. Hakozaki, S. Gonias Murray, J. B. Ling, H. Lassmann, J. L. Degen, M. H. Ellisman, and K. Akassoglou. 2012. Fibrinogen-induced perivascular microglial clustering is required for the development of axonal damage in neuroinflammation. Nat Commun 3: 1227.
Tiller, T., Meffre, S. Yurasov, M. Tsuiji, M. C. Nussenzweig, and H. Wardemann. 2008. Efficient generation of monoclonal antibodies from single human 8 cells by single cell RT-PCR and expression vector cloning. Journal of immunological methods 329:112-124.

### Example II

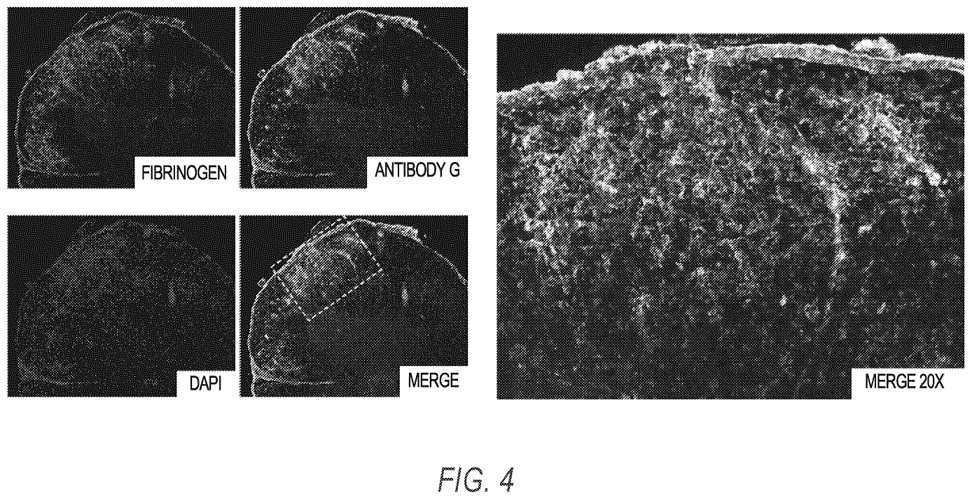

Double IF data on fibrinogen and Antibody G in EAE spinal cords showing fibrinogen target recognition of Ab G. The data show double immunofluorescence with a sheep anti-fibrin(ogen) antibody (Enzyme Research) (red) and antibody G (green) shows antibody G recognition of fibrin(ogen) in the spinal cord. (FIG. 4)

### Example III

### Antibody-G Binding to Fibrinogen and Fibrin

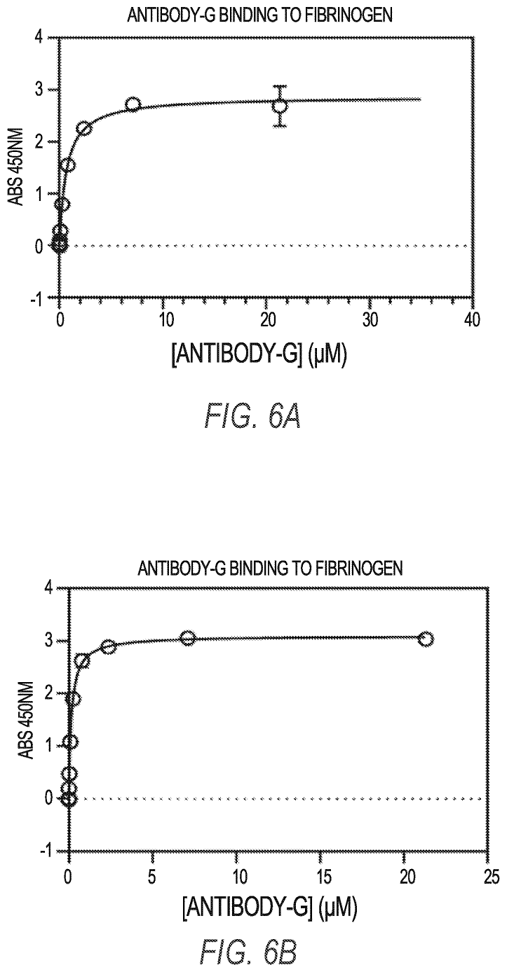

Solid phase ELISA was used to assess whether Antibody G binds to fibrinogen and fibrin separately. Antibody G was incubated for 2 hours at 37° C. on 25 μg/mL IgG-depleted fibrinogen or fibrin in 20 mM HEPES buffer, pH7.2, coated and dried overnight in high protein-binding ELISA plates. Twelve concentrations with 3-fold dilutions of antibody were used to create a dose response curve to calculate dissociation constants (Kd). Antibody G showed a higher affinity for fibrin, with a Kd of 0.16 μM, than to fibrinogen, with a Kd of 0.66 μM (FIG. 6).

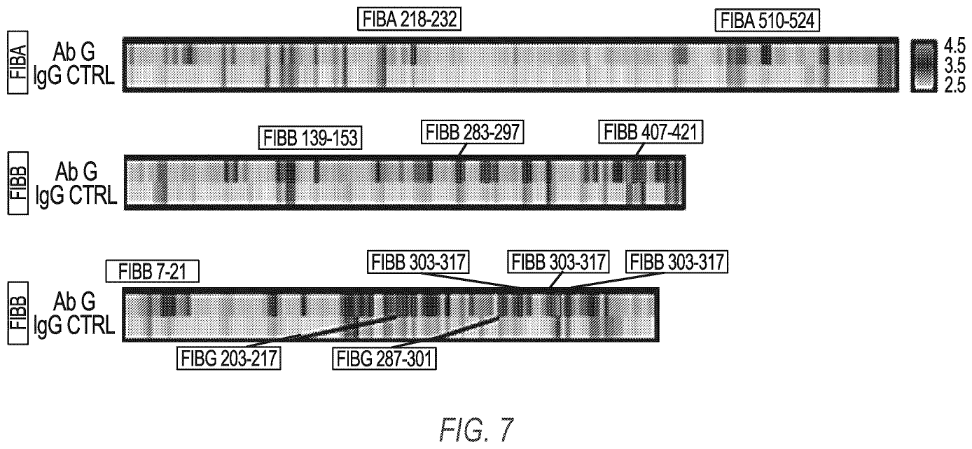

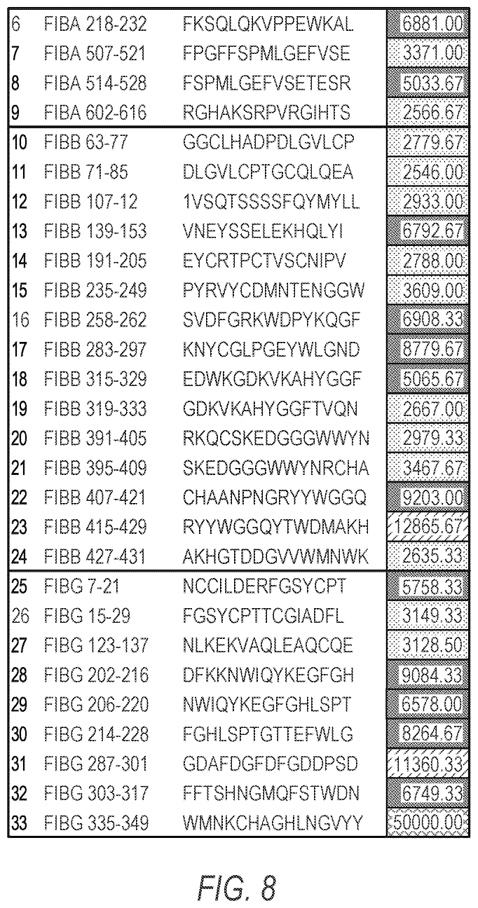

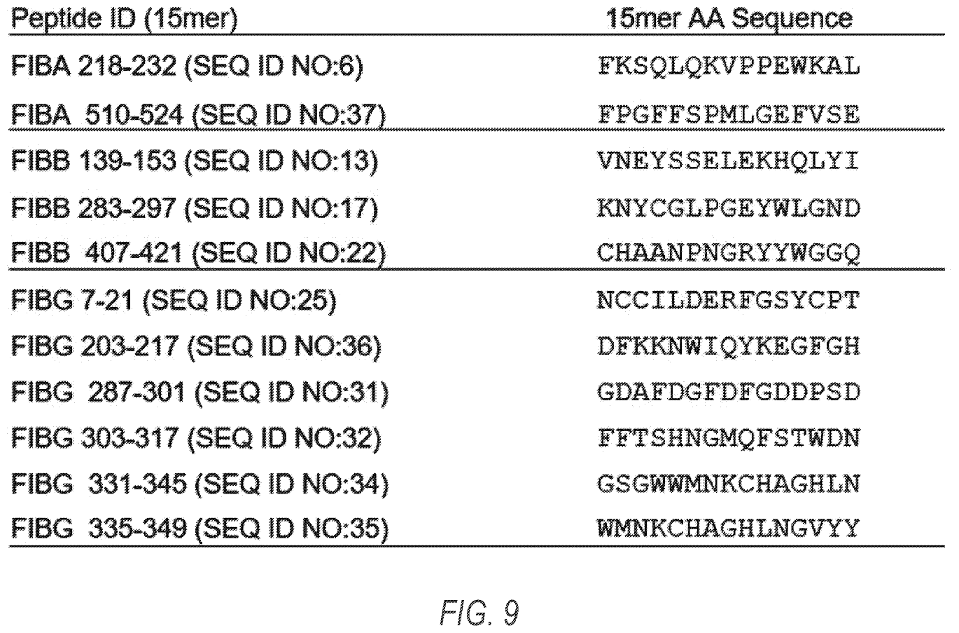

A custom fibrinogen peptide library was generated by printing 15 amino acid length human fibrinogen peptides (15mers) on a microarray (JPT Technologies, Germany). Peptide library microarray was hybridized with 1 ug/mL Antibody G or IgG1 isotype control. Binding was detected by incubation of the microarray with anti-human secondary antibody conjugated with fluorescent dye. Peptide binding was individually detected for each peptide in relative light units (RLUs) and was converted into log 10 scale for data representation (FIG. 7). Peptide binding with log 10(RLU) greater than the Antibody G dataset median value was selected for further background correction to eliminate non-specific binding signal (FIG. 8) (Imholte et al, 2013). Significant specific binding was designated as log 10(background corrected RLU) equal or greater than 1.0 (FIG. 8).

### Antibody-G Epitope Validation

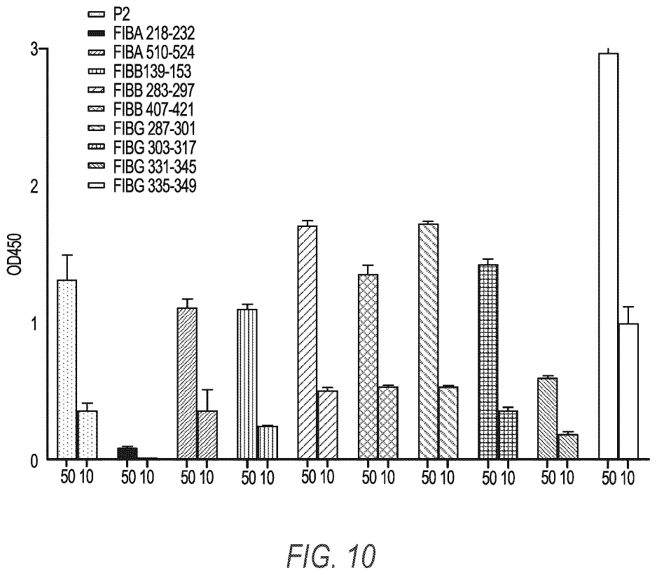

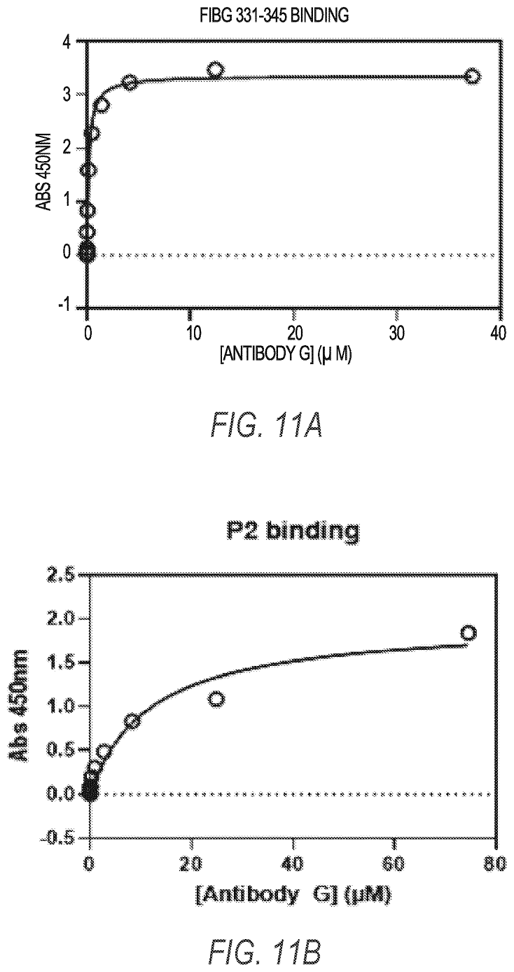

Based on the fibrinogen peptide profiling (FIG. 8), 15mers were selected to carry out individual peptide binding validation using solid phase ELISA. High purity 15mers were synthesized (Genemed) and then each were diluted at 30 μM in 20 mM HEPES buffer, coated in separate wells and dried overnight. Antibody G was incubated on peptide coated wells for 2 hours at 37° C., at a concentration of 25 or 50 μg/mL. Antibody G showed the highest binding to the FIBG 335-349 peptide, lower binding to FIBG 331-345 and little binding to FIBG 287-301 (FIG. 10). Affinity calculations of Antibody G were carried out for select peptide FIBG 331-345 (FIG. 11, left) and compared against P2 peptide (YSMKETTMKIIPFNRLTIG; SEQ ID NO: 1) with Kd of 0.36 μM and 11.8 μM respectively (FIG. 11, right).

### In Vivo Target Engagement of Antibody G in EAE Spinal Cords

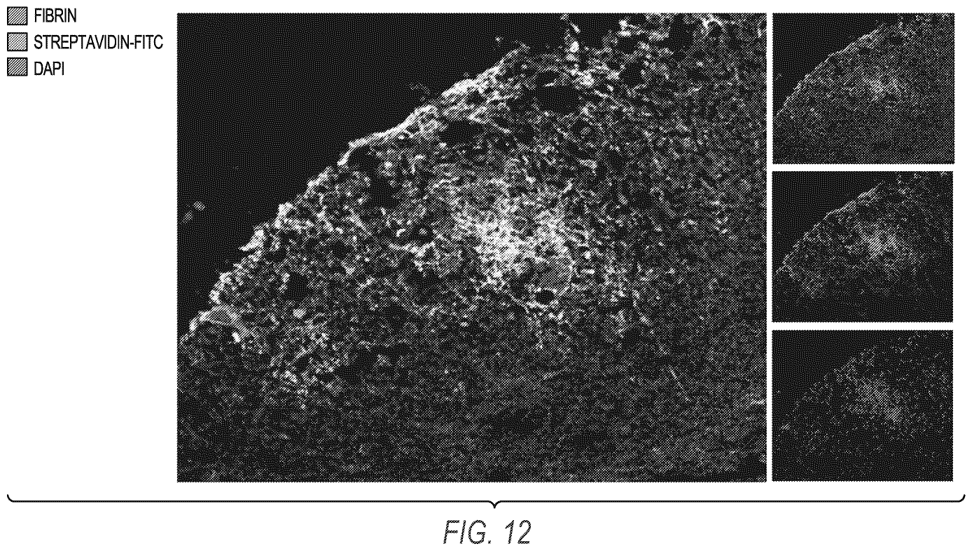

Target recognition and binding to extravascular fibrin was tested in MOG35-55 EAE mice. Biotinylated Antibody G was injected intraperitoneally at 25 mg/kg body weight when clinical score was equal or greater than 3, repeated in 2 days for a total of two injections. Fresh frozen mouse spinal cord tissues were cut into 10-um sections and immunostained with sheep anti-fibrinogen antibody and streptavidin. Images were acquired with an Axioplan II epifluorescence microscope (Zeiss) equipped with dry Plan-Neofluar objective (20×0.5 NA). Extravascular fibrin found in EAE animal spinal cord exhibited significant overlap with Antibody G-Biotin complex in lesions (FIG. 12)

### Target Engagement of Antibody G in EAE Spinal Cord Tissue

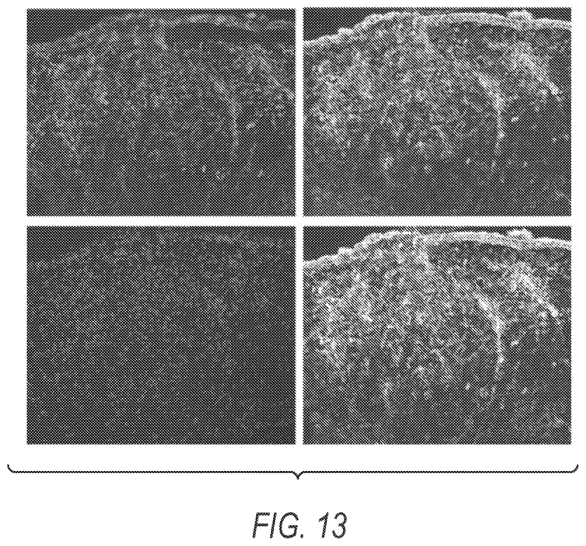

Antibody G immunohistochemistry of MOG35-55 EAE mouse spinal cords were performed along with fibrin immunostaining. Fresh frozen mouse spinal cord tissue was cut into 10-micron sections and immunolabeled with Antibody G (0.4 mg/ml) and anti-fibrinogen antibody. As shown in FIG. 6, the mouse spinal cord from EAE mice showed marked colocalization of Antibody G (green) with fibrin detected in the spinal cord lesions (red) (FIG. 13)

### Antibody G Inhibits Fibrin-Derived Macrophage Activation.

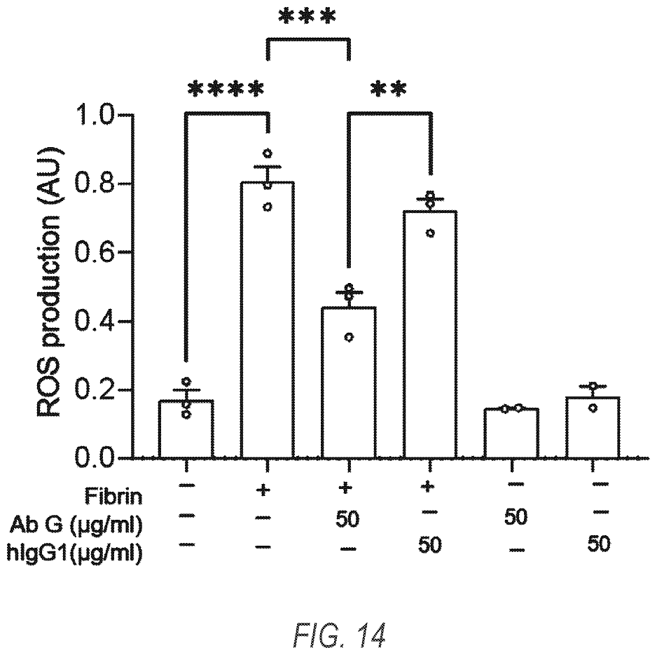

identify the ability of Antibody G to inhibit macrophage activation, a fibrin-induced ROS production was determined using the detection method as described, e.g., in Ryu et al., Nature Immunology (2018). Antibody G was pre-incubated with a fibrin coated plate prior to macrophage plating. Autoantibody presence inhibited fibrin-induced ROS generation at 50 ug/mL. Antibody G exhibited strong inhibition of ROS generation by fibrin as compared with isotype control hIgG1 (FIG. 14).

### Antibody G Inhibits Fibrin Mediated Neuroinflammation.

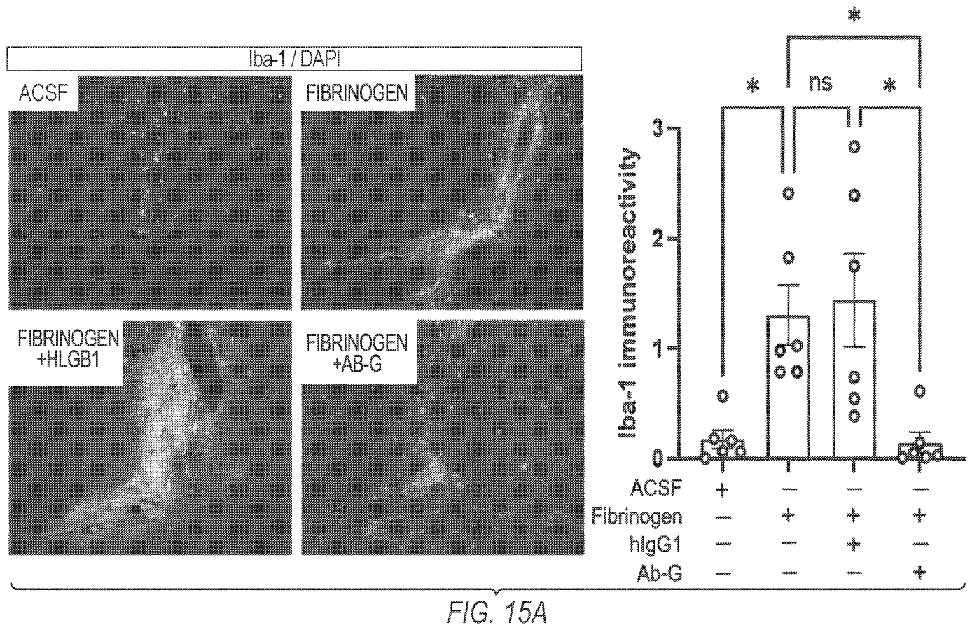

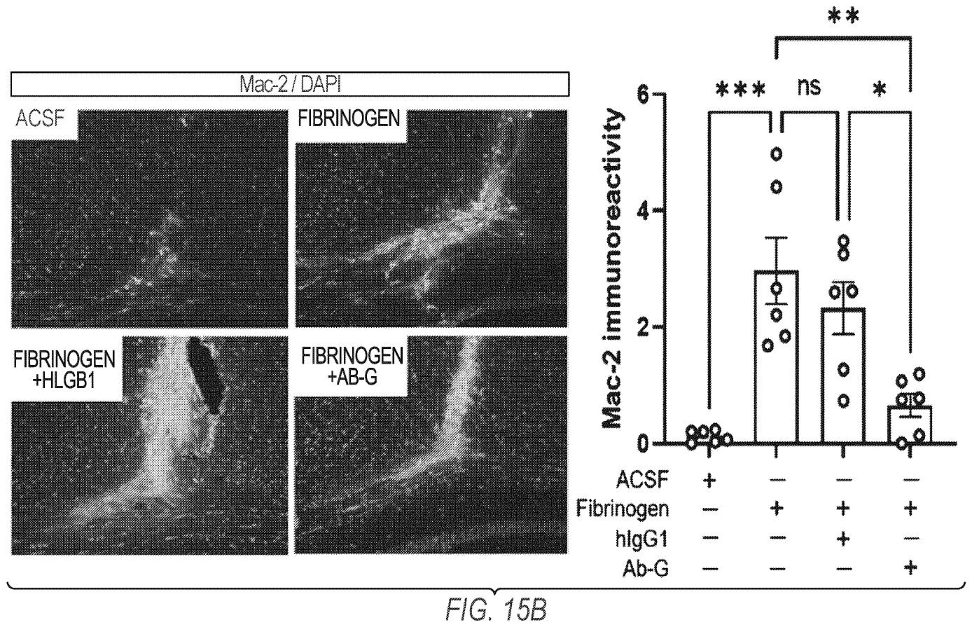

The effect of Ab-G treatment was examined in a fibrinogen-driven mouse model of encephalomyelitis in the corpus callosum as described, e.g., in Ryu et al., Nature Immunology (2018). We investigated the activation of microglia (Iba-1) and infiltrated macrophages (Mac-2) in fibrinogen-injected corpus callosum using immunohistochemistry. Administration of Ab-G blocked microglia activation in the corpus callosum and reduced infiltration of macrophages after fibrinogen injection (FIG. 15A, B).

### Example IV

### Antibody G Reduces Disease Progression and Demyelination in Animal Model of MS

To test the effects of Antibody G in mouse animal models in vivo, we A) generated a murine version of Antibody G (mu-AbG), B) determined its binding to fibrin in biochemical assays and C) administered mu-AbG to the EAE animal model of MS.

### A) Generation of a Murine Version of Antibody G (Mu-AbG)

We generated mu-AbG for the delivery of the antibody in mice for in vivo testing. To generate mu-AbG, we replaced the human IgG1 Fc portion of human Antibody G with murine IgG2a Fc by molecular cloning. The sequences of Murine Antibody G Heavy and Light Chains are shown below:

The nucleotide sequence of murine mAb G- HC (Mouse
IgG2a)
(SEQ ID NO: 38)
GAAGTGCAGCTGGTGGAATCTGGTGGCGGAGCTGTTCAGCCTGGCAGATC
CCTGAGACTGTCTTGTGCTGCTTCCGGCGTGTCCTTCTCCAACATCGGCA
TGCACTGGGTCCGACAGGCCCCTGGAAAAGGATTGGAATGGGTCGCCCTG
ATCTCCTCTGACGGCAGACACACCCACTACGCCGACTCTGTGAAGGGCAG
ATTCACCATCAGCCGGGACAACTCCGAGAACACCCTGTACCTGCAGATCA
ACGGCCTGAGAGCCGATGACACCGCCGTGTACTACTGTGCCAAAGGCCTG
GATACCCGCGCTCGGTACATGGGCAAGTTCTACACCTTCGACTACTGGGG
CCAGGGCACCCTGGTTACAGTTTCTTCTGCTAAGACCACCGCACCTAGTG
TGTACCCTCTGGCTCCTGTGTGTGGCGATACCACAGGCTCCTCTGTGACC
CTGGGATGTCTGGTCAAGGGCTACTTCCCCGAGCCTGTGACACTGACCTG
GAACTCCGGATCTCTGTCCTCCGGCGTGCACACCTTTCCAGCTGTGCTGC
AGTCCGACCTGTACACCCTGTCCAGCTCTGTGACTGTGACCTCCTCCACC
TGGCCTAGCCAGTCCATCACCTGTAACGTGGCCCATCCTGCCTCCAGCAC
CAAGGTGGACAAGAAGATCGAGCCTCGGGGCCCTACCATCAAGCCTTGTC
CTCCATGCAAGTGCCCCGCTCCTAATCTGCTCGGAGGCCCTTCCGTGTTC
ATCTTCCCACCTAAGATCAAGGACGTGCTGATGATCTCCCTGTCTCCTAT
CGTGACCTGCGTGGTGGTGGACGTGTCCGAGGATGATCCTGACGTGCAGA
TTAGTTGGTTCGTGAACAACGTCGAGGTGCACACCGCTCAGACCCAGACA
CACAGAGAGGACTACAACTCCACACTGAGAGTGGTGTCTGCCCTGCCTAT
CCAGCATCAGGATTGGATGTCCGGCAAAGAATTCAAGTGCAAAGTGAACA
ACAAGGACCTGCCTGCTCCAATCGAGCGGACCATCTCTAAGCCTAAGGGC
TCTGTCAGGGCCCCTCAGGTGTACGTTTTGCCACCTCCTGAGGAAGAGAT
GACCAAGAAACAAGTGACCCTGACATGCATGGTCACCGACTTCATGCCCG
AGGACATCTACGTGGAATGGACCAACAACGGCAAGACCGAGCTGAATTAC
AAGAACACAGAGCCCGTGCTGGACTCCGACGGCTCCTACTTCATGTACTC
CAAGCTGCGCGTCGAGAAGAAGAACTGGGTCGAGAGAAACTCCTACTCCT
GCTCCGTGGTGCACGAGGGCCTGCACAATCACCACACCACCAAGTCCTTC
TCTCGGACCCCTGGAAAGTGATGA
The amino acid sequence of murine mAb G HC (Mouse
IgG2a)
(SEQ ID NO: 39)
EVQLVESGGGAVQPGRSLRLSCAASGVSFSNIGMHWVRQAPGKGLEWVAL
ISSDGRHTHYADSVKGRFTISRDNSENTLYLQINGLRADDTAVYYCAKGL
DTRARYMGKFYTFDYWGQGTLVTVSSAKTTAPSVYPLAPVCGDTTGSSVT
LGCLVKGYFPEPVTLTWNSGSLSSGVHTFPAVLQSDLYTLSSSVTVTSST
WPSQSITCNVAHPASSTKVDKKIEPRGPTIKPCPPCKCPAPNLLGGPSVF
IFPPKIKDVLMISLSPIVTCVVVDVSEDDPDVQISWFVNNVEVHTAQTQT
HREDYNSTLRVVSALPIQHQDWMSGKEFKCKVNNKDLPAPIERTISKPKG
SVRAPQVYVLPPPEEEMTKKQVTLTCMVTDFMPEDIYVEWTNNGKTELNY
KNTEPVLDSDGSYFMYSKLRVEKKNWVERNSYSCSVVHEGLHNHHTTKSF
SRTPGK**
The nucleotide sequence of murine mAb G-LC (Mouse
kappa)
(SEQ ID NO: 40)
GATATCCAGCTGACCCAGTCTCCTTCCAGCCTGTCTGCCTCTGTGGGCGA
CAGAGTGACCATCACCTGTCGGGCCTCTCAGAACATCGGCGTGAACCTGA
ACTGGTATCAGCAGAGGCCTGGCAAGGGCCCTAAACTGCTGGTGTCCTCT
ACCTTCAGCCTGCAGTCTGGCGTGCCCTCCAGATTTTCTGCTTCTGGCGC
TGGCACCAACTTTACCCTGACAATCAGCTCCCTGCAGCCTGAGGACTACG
TGACCTACTACTGCCAGCAGTCCTACTCCTCTCCATTCACCTTCGGACCC
GGCACCAAGGTGGACATTAAGAGAGCTGACGCCGCTCCTACCGTGTCTAT
CTTCCCACCCTCTAGCGAGCAGCTGACCTCAGGCGGAGCTTCTGTCGTGT
GCTTCCTGAACAACTTCTACCCCAAGGACATCAACGTGAAGTGGAAGATC
GACGGCTCCGAGAGACAGAACGGCGTGCTGAACTCTTGGACCGACCAGGA
CTCCAAGGACAGCACCTACTCCATGTCCTCCACACTGACCCTGACCAAGG
ACGAGTACGAGCGGCACAACTCCTATACCTGCGAGGCTACCCACAAGACC
TCCACCTCTCCAATCGTGAAGTCCTTCAACCGGAACGAGTGCTGATGA
The amino acid sequence of murine mAb G LC (Mouse
kappa)
(SEQ ID NO: 41)
DIQLTQSPSSLSASVGDRVTITCRASQNIGVNLNWYQQRPGKGPKLLVSS
TFSLQSGVPSRFSASGAGTNFTLTISSLQPEDYVTYYCQQSYSSPFTFGP
GTKVDIKRADAAPTVSIFPPSSEQLTSGGASVVCFLNNFYPKDINVKWKI
DGSERQNGVLNSWTDQDSKDSTYSMSSTLTLTKDEYERHNSYTCEATHKT
STSPIVKSENRNEC**
**Constant region
No signal sequence is included.

### B) Murine Antibody G Binding to Fibrin

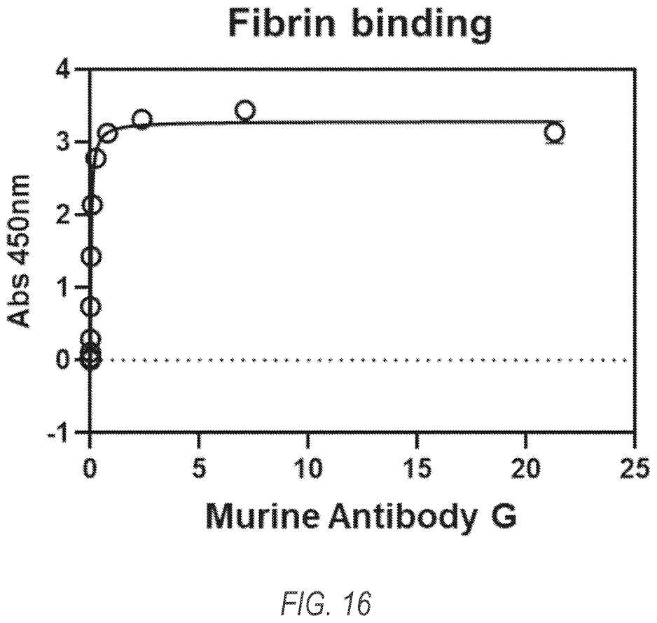

Solid phase ELISA was used to assess murine Antibody G binding to fibrin. Antibody G was incubated for 2 hours at 37° C. on 25 μg/mL IgG-depleted fibrin in 20 mM HEPES buffer, pH7.2, coated and dried overnight in high protein-binding ELISA plates. Twelve concentrations with 3-fold dilutions of antibody were used to create a dose response curve to calculate dissociation constants (Kd). Murine Antibody G showed a higher affinity for fibrin, with a Kd of 0.04 μM (FIG. 16), than human Antibody G, with a Kd of 0.16 μM (FIG. 6).

### C) Administration of Murine Antibody G in EAE

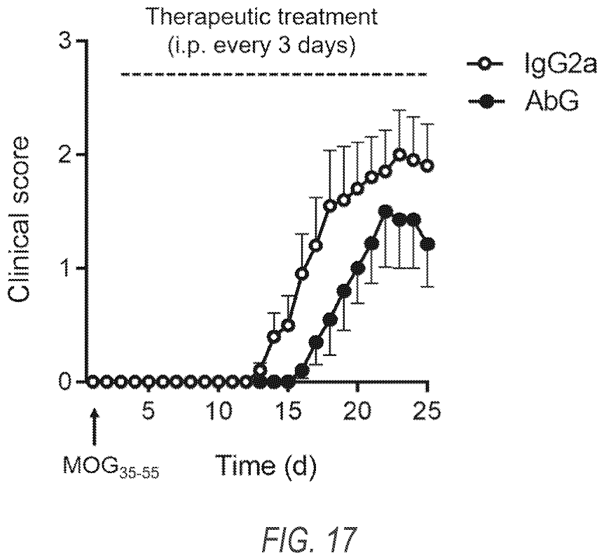

To test the effects of Antibody G to mitigate disease symptoms and/or progression in vivo, an experimental model involving EAE induction in C57bl/6 mice using myelin oligodendrocyte glycoprotein amino acids 35-55 (MOG35-55) was employed. In this study, we investigated the effects of autoantibody Antibody-G (mu-AbG) compared to an isotype control IgG2a. Antibody administration started three days after immunization with MOG. Antibodies were delivered via intraperitoneal injection every three days over a 20-day period. Mice treated with mu-AbG exhibited notably milder neurological symptoms in contrast to those treated with IgG2a, particularly during the initial-to-peak phase of the disease progression (FIG. 17). Throughout the entire treatment duration following EAE induction, the mu-AbG treatment consistently suppressed clinical severity of the disease in comparison to the mice treated with IgG2a. This underscores the therapeutic potential of AbG administration, as it holds the capacity to mitigate disease severity and progression within this EAE model, thereby offering valuable therapeutic benefits.

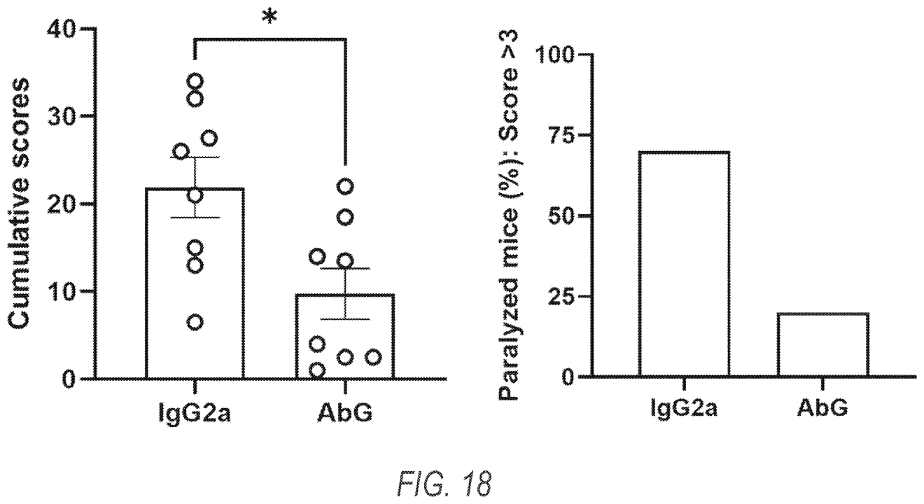

For a more comprehensive assessment of the impact of mu-AbG antibody treatment on disease severity, an in-depth analysis was conducted by evaluating the cumulative disease scores of the mice along with the percentage of mice that experienced paralysis within each treatment group (FIG. 18). Notably, administering 40 mg/kg of mu-AbG to MOG-induced EAE mice resulted in a significant reduction in cumulative disease scores, as clearly illustrated in the left graph of FIG. 18.

Furthermore, the administration of mu-AbG exhibited a distinct effect in mitigating the induction of paralysis among EAE mice, as illustrated in the right graph of FIG. 18. These findings collectively suggest that the anti-fibrin antibody mu-AbG originally derived from MS patients plays a pivotal role in diminishing disease severity following treatment. This not only confirms the therapeutic potential of AbG but also highlights its capacity to contribute to the amelioration of disease-associated symptoms, further supporting its candidacy for future therapeutic interventions.

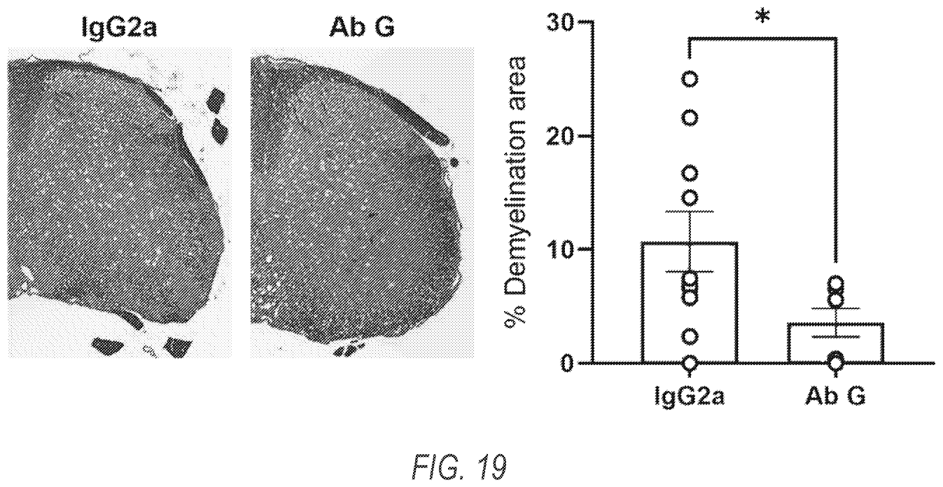

To investigate the potential impact of AbG on myelin damage throughout the disease progression (FIG. 19), we conducted a detailed histological assessment. Specifically, we employed histochemical analysis, utilizing luxol fast blue staining, in order to examine the spinal cord tissue of EAE mice that received either mu-AbG or IgG2a treatment. The white matter regions of spinal cord sections from mu-AbG-treated EAE mice (as shown in the left image panel of FIG. 19), a robust and noticeable preservation of myelin was observed. Furthermore, to quantify the extent of demyelination, we performed an analysis of the demyelinated areas. As shown in the right graph of FIG. 19, the results indicated that mice treated with mu-AbG exhibited notably greater protection against EAE-induced demyelination compared to those treated with IgG2a.

These findings strongly suggest that the AbG antibody protects from myelin damage in the context of inflammatory demyelinating diseases. This underscores AbG's therapeutic potential, emphasizing its ability to counteract the damaging effects of the disease process on myelin integrity. To our knowledge, these results show for the first-time protective autoantibodies derived from MS patients that effectively guard against inflammatory demyelination driven by fibrin-derived processes in an MS model.

All publications, nucleotide and amino acid sequence identified by their accession nos., patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

The specific methods and compositions described herein are representative of embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims. As used herein and in the appended claims, the singular forms “a,” “an,” and “the” include plural reference unless the context clearly dictates otherwise. Thus, for example, a reference to “a nucleic acid” or “a polypeptide” includes a plurality of such nucleic acids or polypeptides (for example, a solution of nucleic acids or polypeptides or a series of nucleic acid or polypeptide preparations), and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

1. A human antibody or fragment thereof comprising a heavy chain variable domain (VH) comprising the amino acid sequence of SEQ ID NO:3 or at least 95% identity thereto and a light chain variable domain (VL) comprising the amino acid sequence of SEQ ID NO:5 or at least 95% identity thereto.

2. The antibody or fragment thereof of claim 1, wherein the antibody or fragment thereof comprises of a heavy chain of IgG, IgA, IgD, IgE or IgM.

3. The antibody or fragment thereof of claim 1, wherein the antibody or fragment thereof comprises of a Fc region of IgG including wild type or mutant subclasses IgG1, IgG2, IgG3 and IgG4.

4. The antibody or fragment thereof of claim 1, wherein the antibody or fragment thereof can bind fibrin and/or fibrinogen.

5. The antibody or fragment thereof of claim 1, wherein the antibody or fragment thereof binds one or more of SEQ ID NOs: 1, 6, 13, 17, 22, 25, 31, 32, 34, 35, 36 or 37.

6. The antibody or fragment thereof of claim 1, wherein the antibody or fragment thereof inhibits proinflammatory properties of fibrin in macrophages.

7. The antibody or fragment thereof of claim 1, wherein the antibody or fragment thereof inhibits proinflammatory properties of fibrin in microglia.

8. The antibody or fragment thereof of claim 1, wherein the antibody or fragment thereof does not affect blood coagulation.

9. A composition comprising the antibody or fragment thereof of claim 1 and a carrier.

10. A method to treat or prevent a neurodegenerative disease or disorder comprising administering to a subject in need thereof an effective amount of the antibody or fragment thereof of claim 1.

11. The method of claim 10, wherein the neurodegenerative disease or disorder is selected from the group consisting of multiple sclerosis (MS), progressive multifocal leukoencephalopathy (PML), encephalomyelitis (EPL), central pontine myelolysis (CPM), adrenoleukodystrophy, Alexander's disease, Pelizaeus Merzbacher disease (PMZ), Wallerian Degeneration, optic neuritis, transverse myelitis, amyotrophic lateral sclerosis (ALS), Huntington's disease, Alzheimer's disease, Parkinson's disease, spinal cord injury, traumatic brain injury, post radiation injury, neurologic complications of chemotherapy, stroke, acute ischemic optic neuropathy, vitamin E deficiency, isolated vitamin E deficiency syndrome, AR, Bassen-Kornzweig syndrome, Marchiafava-Bignami syndrome, metachromatic leukodystrophy, trigeminal neuralgia, acute disseminated encephalitis, seizures, Guillian-Barre syndrome, Marie-Charcot-Tooth disease and Bell's palsy.

12. A method to treat a condition/disease in which fibrin plays a role comprising administering to a subject in need thereof an effective amount of the antibody or fragment thereof of claim 1 or the composition of claim 9 including, wherein the condition/disease comprises inflammation, periodontal disease, rheumatoid arthritis (RA), cancer, colitis or muscular dystrophy.

13. A method to treat coronavirus comprising administering to a subject in need thereof an effective amount of the antibody or fragment thereof of claim 1, wherein the subject is infected with SARS-CoV-2 or SARS-CoV-1.

14. A method to inhibit microglial activating comprising administering to a subject in need thereof an effective amount of the antibody or fragment thereof of claim 1.

15. A method to diagnose a fibrin-related disease or disorder comprising:
(a) contacting the antibody or fragment thereof of claim 1 with a sample from a subject under conditions that allow polypeptide/antibody complexes to form; and
(b) detecting polypeptide/antibody complexes of a), wherein the detection of polypeptide/antibody complexes is an indication that fibrin is present in the sample;
(c) conjugating the antibody with a radioligand for non-invasive detection of fibrin in tissues (Fibrin-PET).

16. A kit comprising the antibody or fragment thereof of claim 1.

17. A method to promote remyelination comprising administering to a subject in need thereof the antibody or fragment thereof of claim 1.

18. A method to protect a subject from myelin damage or inhibit myelin damage in a subject comprising administering to said subject the antibody or fragment thereof of claim 1, wherein the myelin damage is due to an inflammatory demyelinating disease.
